(19) United States
(12) Patent Application Publication
(10) Pub. No.: US 2001/0034042 A1
(43) Pub. Date: Oct. 25, 2001

(54) Complexes of peptide-binding fragments of heat shock proteins and their use as immunotherapeutic agents

(76) Inventor: Pramod K. Srivastava, Avon, CT (US)

Correspondence Address: PENNIE AND EDMONDS, 1155 AVENUE OF THE AMERICAS, NEW YORK, NY, 100362711

(21) Appl. No.: 09/759,010

(22) Filed: Jan. 12, 2001

Related U.S. Application Data
(63) Continuation-in-part of application No. 09/488,393, filed on Jan. 20, 2000.

(51) Int. Cl. C12P 21/06; A61K 38/17

(52) U.S. Cl. 435/68.1; 514/12

(57) ABSTRACT

The present invention relates to pharmaceutical compositions comprising peptide-binding fragments of heat shock proteins (HSPs) and noncovalent complexes of peptide-binding fragments of HSPs in noncovalent association with antigenic molecules. The invention further relates to methods for the use of such pharmaceutical compositions as immunotherapeutic agents for the treatment and prevention of infectious diseases and cancer.

This is a continuation-in-part of U.S. Patent application Ser. No. 09/488,393, filed Jan. 20, 2000, which is incorporated by reference herein in its entirety.

This invention was made with government support under grant numbers CA44786 and CA64394 awarded by the National Institutes of Health. The government has certain rights in the invention.

### TABLE OF CONTENTS

1. INTRODUCTION 1

2. BACKGROUND OF THE INVENTION 1

2.1. Heat Shock Proteins 1

2.2. Immunogenicity of Heat Shock/Stress Proteins 2

3. SUMMARY OF INVENTION 4

4. BRIEF DESCRIPTION OF THE DRAWINGS 11

5. DETAILED DESCRIPTION OF THE INVENTION 12

5.1 HSP Peptide-Binding Domain Fragments 15

5.2 Preparation of HSP Peptide-Binding Fragments 22

5.2. 1. Production of Recombinant HSP Peptide-Binding Fragments 23

5.2.1.1 Isolation of HSP Gene Sequences 23

5.2.1.2 Recombinant Expression 29

5.2.1.3 Purification Methods for Recombinant HSP Peptide-Binding Fragments 33

5.2.1.4 Host-Vector Systems 36

5.2.2 Synthetic Production of HSP Peptide-Binding Fragments 39

5.2.3 Production of HSP Peptide-binding Fragments by Proteolytic Cleavage 40

5.3 HSP Peptide-binding Fragments - Antigenic Molecule Complexes 41

5.3.1 Preparation of Intracellular Complexes of HSP Peptide-Binding Fragments with Antigenic Molecules 41

5.3.1.1 Preparation of Hsp70-Peptide Complexes 43

5.3.1.2 Preparation of Hsp90-Peptide Complexes 45

5.3.1.3 Preparation of gp96-Peptide Complexes 46

5.3.2 In Vitro Complexing 48

5.3.3 Sources of Antigenic Molecules 50

5.3.3.1 Exogenous Antigenic Molecules 50

5.3.3.2 Peptides From Hsp-Peptide Complexes 52

5.3.3.3 Peptides from MHC-Peptide Complexes 52

5.3.3.4 Synthetic Peptides 53

5.3.3.5 Recombinantly Produced Antigens 53

5.4 Therapeutic Applications of Noncovalent Complexes of HSP Peptide-Binding Fragments and Antigenic Molecules 54

5.4.1 Prevention and Treatment of Infectious Diseases 54

5.4.2 Prevention and Treatment of Cancer 56

5.4.3 Combination With Adoptive Immunotherapy 59

5.4.3.1 Sensitization of Macrophages and Antigen Presenting Cells with HSP Peptide-Binding Fragment-Peptide Complexes 60

5.4.3.2 Reinfusion of Sensitized APC 61

5.4.5 Monitoring of Effects During Immunotherapy 63

5.4.5.1 Delayed Hypersensitivity Skin Test 63

5.4.5.2 Activity of Cytolytic T Lymphocytes In Vitro 63

5.4.5.3 Levels of Tumor Specific Antigens 64

5.4.5.4 Computed Tomographic (CT) Scan 64

5.4.5.5 Measurement of Putative Biomarkers 65

5.4.5.6 Sonogram 65

5.5 Dosage Regimens 65

5.6 Formulation 67

6. EXAMPLE: Administration of Hsp Peptide-Binding Fragment-Peptide Complexes in the Treatment of Infectious Disease 70

7. EXAMPLE: Adoptive Transfer of Sensitized Macrophage, in Combination with Administration of Peptide-Binding HSP Peptide-Binding Fragment-Peptide Complexes 71

7.1. Material and Methods 71

7.2. Treatment of Hepatocellular Carcinoma 72

### 1. INTRODUCTION

The present invention relates to pharmaceutical compositions comprising peptide-binding fragments of heat shock proteins (HSPs) and peptide-binding fragments of HSPs in noncovalent association with antigenic molecules. The invention further relates to methods for the use of such pharmaceutical compositions as immunotherapeutic agents for the treatment and prevention of infectious diseases and cancer.

### 2. BACKGROUND OF THE INVENTION

2.1. Heat Shock Proteins

Heat shock proteins (HSPs), also referred to interchangeably as stress proteins, were first identified as proteins synthesized by a cell in response to heat shock. To date, five major classes of HSPs have been identified, based on the molecular weight of the family members. These classes are called sHSPs (small heat shock proteins), Hsp60, Hsp70, Hsp90, and Hsp100, where the numbers reflect the approximate molecular weight of the HSPs in kilodaltons.

Many HSPs have been found to be induced in response to stressful stimuli other than heat, including nutrient deprivation, metabolic disruption, oxygen radicals, and infection with intracellular pathogens (see Welch, May 1993, Scientific American 56-64; Young, 1990, Annu. Rev. Immunol. 8:401-420; Craig, 1993, Science 260:1902-1903; Gething et al., 1992, Nature 355:33-45; and Lindquist et al., 1988, Annu. Rev. Genetics 22:631-677).

HSPs are involved not only in cellular protection against adverse conditions, but are also involved in essential biochemical and immunological processes in unstressed cells. For example, HSPs are involved in various kinds of chaperoning functions. Members of the Hsp70 family, located in the cell cytoplasm, nucleus, mitochondria, or endoplasmic reticulum are involved in the presentation of antigens to the cells of the immune system, and are also involved in the transfer, folding and assembly of proteins in normal cells (Lindquist et al., 1988, Ann. Rev. Genetics 22:631-677). A number of proteins thought to be involved in chaperoning are residents of the endoplasmic reticulum (ER) lumen, for example, protein disulfide isomerase (PDI; Gething et al., 1992, Nature 355:33-45), Grp94 or ERp99 (Sorger and Pelham, 1987, J. Mol. Biol., 194:991-94) which is related to Hsp90, and Grp78 or BiP, which is related to Hsp70 (Munro et al., 1986, Cell 46:291-300; Haas & Webl, 1983, Nature 306:387-389). These proteins are known to bind a variety of mutant, unfolded, incompletely glycosylated proteins (Machamer et al., 1990, J. Biol. Chem. 65:6879-6883; Gething et al., 1986, Cell 46:939-950).

Heat shock proteins bind polypeptides for their various cellular activities. The binding and release of polypeptides is facilitated by adenosine triphosphate (ATP), at least in the Hsp70 and Hsp90, and possibly other, families of proteins. ATP hydrolysis accompanies polypeptide binding, followed by the subsequent release of the polypeptide in Hsp70 (Flynn et al., 1989, Science, 245:385-390) and Hsp90 (Obermann et al., J. Cell. :z 15;: Biol., 1998,143:901-10). Structural analysis of DnaK, the Hsp70 family protein of E. coli, suggests that a conformational change in the protein structure is associated with this ATP and peptide binding cycle (Zhu et al, 1996, Science 272:1606-14).

In addition to the classical heat shock and stress responsive proteins, the Hsp60, Hsp70 and Hsp90 families are also composed of proteins that are related to the HSPs in sequence, having greater than 35% amino acid identity, but whose expression levels are not altered by heat shock or stress.

Heat shock proteins are among the most highly conserved proteins in existence. For example, DnaK, the Hsp70 from E. coli has about 50% amino acid sequence identity with Hsp70 proteins from excoriates (Bardwell et al., 1984, Proc. Natl. Acad. Sci. 81:848-852). The Hsp60 and Hsp90 families also show similarly high levels of intra-family conservation (Hickey et al., 1989, Mol. Cell. Biol. 9:2615-2626; Jindal, 1989, Mol. Cell. Biol. 9:2279-2283).

2.2. Immunogenicity of Heat Shock/Stress Proteins

Srivastava et al. demonstrated immune response to methylcholanthrene-induced sarcomas of inbred mice (1988, Immunol. Today 9:78-83). In these studies, it was found that the molecules responsible for the individually distinct immunogenicity of these tumors were identified as cell-surface glycoproteins of 96 kDa (gp96) and intracellular proteins of 84 to 86 kDa (Srivastava et al., 1986, Proc. Natl. Acad. Sci. USA 83:3407-3411; Ullrich, S. J. et al., 1986, Proc. Natl. Acad. Sci. USA 83:3121-3125). Immunization of mice with gp96 or p84/86 isolated from a particular tumor rendered the mice immune to that particular tumor, but not to antigenically distinct tumors. Isolation and characterization of genes encoding gp96 and p84/86 revealed significant homology between them, and showed that gp96 and p84/86 were, respectively, the endoplasmic reticular and cytosolic counterparts of the same heat shock proteins (Srivastava et al., 1988, Immunogenetics 28:205-207; Srivastava et al., 1991, Curr. Top. Microbiol. Immunol. 167:109-123). Further, Hsp70 was shown to elicit immunity to the tumor from which it was isolated but not to antigenically distinct tumors. However, Hsp70 depleted of peptides was found to lose its immunogenic activity (Udono and Srivastava, 1993, J. Exp. Med. 178:1391-1396). These observations suggested that the heat shock proteins are not immunogenic per se, but form noncovalent complexes with antigenic peptides, and the complexes can elicit specific immunity to the antigenic peptides (Srivastava, 1993, Adv. Cancer Res. 62:153-177; Udono et al., 1994, J. Immunol., 152:5398-5403; Suto et al, 1995, Science, 269:1585-1588).

The use of noncovalent complexes of stress proteins and peptides, purified from cancer cells, for the treatment and prevention of cancer, as well as the use of such complexes in combination with adoptive immunotherapy, has been described (see U.S. Pat. No. 5,750,199; U.S. Pat. No. 5,830,464; Patent Cooperation Treaty (“PCT”) publications WO 96/10411, dated Apr. 11, 1996; and WO 97/10001, dated Mar. 20, 1997; each of which is incorporated by reference herein in its entirety. The purification of stress protein-peptide complexes from cell lysates has been described previously; stress protein-peptide complexes can be isolated from pathogen-infected cells and used for the treatment and prevention of infection caused by pathogens, such as viruses and other intracellular pathogens, including bacteria, protozoa, fungi and parasites (see PCT publication WO 95/24923, dated Sep. 21, 1995).

Immunogenic stress protein-peptide complexes can also be prepared by in vitro complexing of stress protein and antigenic peptides, and the uses of such complexes for the treatment and prevention of infectious diseases and cancer has been described in PCT publication WO 97/10000, dated Mar. 20, 1997. The use of heat shock proteins in combination with a defined antigen for the treatment of infectious diseases and cancer have also been described in PCT publication WO 97/06821, dated Feb. 27, 1997. The administration of expressible polynucleotides encoding eukaryotic heat shock proteins to mammalian cells for stimulating an immune response, and for treatment of infectious diseases and cancer has been described in PCT publications, WO 97/06685 and WO 97/06828, both dated Feb. 27, 1997. The use of stress protein-peptide complexes for sensitizing antigen presenting cells in vitro for use in adoptive immunotherapy is described in PCT publication WO 97/10002, dated Mar. 20, 1997.

These references, however, have not identified portions of HSPs that could be effective in the treatment of infectious disease and cancer. Methods for identifying and producing such HPBFs would facilitate more efficient methods for producing HSPs and HSP-peptide complexes.

Citation or discussion of a reference herein shall not be construed as an admission that such is prior art to the present invention.

### 3. SUMMARY OF INVENTION

The present invention relates to pharmaceutical compositions comprising peptide-binding fragments of heat shock proteins (HSPs) and complexes of peptide-binding fragments of HSPs in noncovalent association with antigenic molecules. The invention further relates to methods for the use of such pharmaceutical compositions as immunotherapeutic agents for the prevention and treatment of infectious diseases and cancer.

In one embodiment, the invention provides a pharmaceutical composition comprising an amount of a molecular complex effective for treatment or prevention of an infectious disease or cancer, said molecular complex comprising a heat shock protein fragment noncovalently associated with an antigenic molecule and a pharmaceutically acceptable carrier, said heat shock protein fragment comprising that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, and wherein said antigenic molecule displays the antigenicity of an antigen of an infectious agent or of a cancer cell. In specific embodiment, the heat shock protein of such a pharmaceutical composition is Hsp70, Hsp90, gp96, calreticulin, or PDI. In another embodiment, said heat shock protein fragment lacks one or more other domains of the heat shock protein.

In another embodiment, the invention provides a recombinant cell infected with a pathogen and transformed with a nucleic acid comprising a nucleotide sequence that (i) is operably linked to a promoter, and (ii) encodes a heat shock protein fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, which heat shock protein fragment noncovalently associates with an antigenic molecule when said antigenic molecule is present, to form a complex that in sufficient amount is capable of eliciting an immune response to the antigenic molecule. In one embodiment, said heat shock protein fragment lacks one or more other domains of the heat shock protein. In a specific embodiment, such a recombinant cell is a human cell.

The invention further provides a recombinant cancer cell transformed with a nucleic acid comprising a nucleotide sequence that (i) is operably linked to a promoter, and (ii) encodes a heat shock protein fragment comprising a peptide-binding domain, which heat shock protein fragment noncovalently associates with an antigenic molecule when said antigenic molecule is present to form a complex that in sufficient amount is capable of eliciting an immune response to the antigenic molecule. In one embodiment, said heat shock protein fragment lacks one or more other domains of the heat shock protein. In a specific embodiment, the cancer cell is a human cell.

In another embodiment, the invention provides a recombinant cell transformed with (i) a first nucleic acid comprising a first nucleotide sequence that is operably linked to a first promoter and that encodes a heat shock protein fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, and (ii) a second nucleic acid comprising a second nucleotide sequence that is operably linked to a second promoter and encodes an antigenic molecule, such that the heat shock protein fragment and the antigenic molecule are expressed within the cell and noncovalently associate with each other to form a complex that in sufficient amount is capable of eliciting an immune response to the antigenic molecule.

The invention further provides pharmaceutical compositions comprising recombinant cells, as described above, and a pharmaceutically acceptable carrier.

The invention further encompasses methods for preparation of noncovalent complexes of HSP peptide-binding fragments and antigenic peptide molecules. In one embodiment, the invention provides a method for preparing a complex of a heat shock protein peptide-binding fragment noncovalently associated with a peptide, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, comprising: a) culturing cells, transformed with a nucleic acid comprising a nucleotide sequence encoding the heat shock protein fragment and operably linked to a promoter, under conditions such that the heat shock protein fragment is expressed by the cells and associates with peptides of the cells; and b) recovering a population of complexes of the heat shock protein fragment noncovalently associated with peptides from the host cell. In one embodiment, the heat shock protein fragment of said method lacks one or more other heat shock protein domains.

The invention further provides a method for preparing a heat shock protein fragment noncovalently associated with peptides derived from one or more antigens of an infectious agent, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, comprising: a) culturing infected cells transformed with a nucleic acid comprising a nucleotide sequence encoding the heat shock protein peptide-binding fragment and operably linked to a promoter under conditions such that the heat shock protein peptide-binding fragment is expressed by the cells and associates with peptides of the cells; and b) recovering a population of complexes of the heat shock protein peptide-binding fragment and peptides derived from the infectious antigen.

In another embodiment, the invention further provides a method for preparing a complex of a heat shock protein fragment noncovalently associated with a peptide, said heat shock protein fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said method comprising digesting a preparation of heat shock proteins noncovalently associated with peptides with a protease under conditions and for a length of time sufficient for the formation of fragments of the heat shock protein noncovalently associated with peptides.

In another embodiment, the invention further provides a method for preparing a complex of a heat shock protein fragment noncovalently associated with a peptide, said heat shock protein fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said method comprising: a) digesting a preparation of heat shock proteins with a protease under conditions and for a length of time sufficient for the formation of fragments of the heat shock protein; and b) contacting the fragments with peptides under conditions and for a length of time sufficient for the formation of complexes of heat shock protein peptide-binding fragments noncovalently associated with peptides.

In specific embodiments, the methods for preparation of such HPBF-peptide complexes further comprise purifying the complexes. In another specific embodiment, the methods further comprise purifying the complexes by affinity chromatography.

The invention further provides a method for preparing in vitro complexes of heat shock protein peptide-binding fragments noncovalently associated with one or more antigenic molecules, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said method comprising incubating a heat shock protein fragment and one or more antigenic molecules under conditions and for a length of time sufficient for the formation of the complexes. In one embodiment of this method, one or more antigenic molecules is a population of peptides from an infected cell or a cancer cell. In another embodiment of this method, one or more antigenic molecules display(s) the antigenicity of an antigen of an infectious agent or a cancer cell. The invention further encompasses the use of heat shock protein peptide-binding fragments in methods for eliciting an immune response. In various embodiments, using in vivo and in vitro techniques, noncovalent complexes of heat shock protein peptide-binding fragments with antigenic molecules are produced. Such complexes may then be used to elicit an immune response by administering an effective amount of complex of HSP peptide-binding fragment bound to an antigenic molecule.

In one embodiment, the invention provides a method of eliciting an immune response against an antigen in an individual comprising administering to the individual an immunogenic complex of a heat shock protein fragment noncovalently associated with a first antigenic molecule displaying antigenicity of the antigen, said heat shock protein fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400. In one embodiment, heat shock protein fragment of said method lacks one or more other heat shock protein domains. In another embodiment, the invention provides the above-described method, further comprising, before, concurrently, or after administration of the immunogenic complex, administering to the individual a composition comprising antigen presenting cells sensitized in vitro with a sensitizing amount of a second immunogenic complex consisting essentially of a heat shock protein, or fragment thereof, noncovalently bound to a second antigenic molecule, in which said second antigenic molecule shares at least one antigenic determinant with the first antigenic molecule.

The invention further encompasses the use of heat shock protein peptide-binding fragments in methods for immunotherapy for treatment and prevention of infectious diseases. In various embodiments, heat shock protein peptide-binding fragments are used in methods for preventing or treating an infectious disease in an individual having an infectious disease, or in whom prevention of an infectious disease is desired. In various embodiments the infectious disease is caused by an infectious agent selected from the group consisting of viruses, bacteria, fungi, and parasites.

In one embodiment, a method is provided comprising administering to the individual an immunogenic complex of a heat shock protein peptide-binding fragment noncovalently associated with a first antigenic molecule, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, wherein the first antigenic molecule displays the antigenicity of an antigen of an infectious agent of the infectious disease. In one embodiment, the heat shock protein fragment of said method lacks one or more other heat shock protein domains. In another embodiment, the method further comprises, before, concurrently or after administration of the immunogenic complex, administering to the individual a composition comprising antigen presenting cells sensitized in vitro with a sensitizing amount of a second complex of a heat shock protein or fragment thereof noncovalently bound to a second antigenic molecule, said second antigenic molecule sharing at least one antigenic determinant with the first antigenic molecule.

In another embodiment, the invention further provides a method of treating or preventing an infectious disease in a subject having an infectious disease or in whom prevention of an infectious disease is desired comprising: a) culturing an infected cell transformed with a nucleic acid comprising a nucleotide sequence encoding a heat shock protein peptide-binding fragment, said infected cell displaying the antigenicity of an antigen of an infectious agent of the infectious disease, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said nucleotide sequence being operably linked to a promoter, under conditions such that the fragment is expressed by the infected cells and associates with peptides of the cell; b) recovering complexes of the heat shock protein fragments noncovalently associated with peptides from the infected cell; and c) administering to the subject an amount of the recovered complexes effective to treat or prevent the infectious disease. In another embodiment, the method further comprises prior to step (a) the step of obtaining infected cells from the subject and transforming the infected cells with the nucleic acid. In another embodiment, the method further comprises prior to step (a) the step of obtaining the infected cell from one or more individuals and transforming the infected cells with the nucleic acid, said one or more individuals being different from the subject and having the same type of infectious disease as the subject. In one embodiment, the heat shock protein fragment of said method lacks one or more other heat shock protein domains.

The invention further encompasses the use of heat shock protein peptide-binding fragments in methods for immunotherapy for treatment and prevention of cancer. In various embodiments, heat shock protein peptide-binding fragments are used in methods to prevent or inhibit growth of a tumor or treat an individual with cancer. In one embodiment a method is provided for treating or preventing cancer in an individual having a type of cancer or in whom prevention of a type of cancer is desired comprising administering to the individual an immunogenic complex of a heat shock protein fragment noncovalently associated with a first antigenic molecule, said heat shock protein comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, wherein either (a) the first antigenic molecule displays antigenicity of said type of cancer or a metastasis thereof; or (b) the complex is obtained by recovering complexes from said type of cancer cells or a metastasis thereof that recombinantly express the heat shock protein fragment. In another embodiment, this method further comprises, before, concurrently or after administration of the immunogenic complex, administering to the individual a composition comprising antigen presenting cells sensitized in vitro with a sensitizing amount of a second complex of a heat shock protein or fragment thereof noncovalently bound to a second antigenic molecule, said second antigenic molecule sharing at least one antigenic determinant with the first antigenic molecule.

In another embodiment, the invention provides a method of treating or preventing cancer in a subject having a type of cancer or in whom prevention of a type of cancer is desired comprising: a) culturing a cancer cell transformed with a nucleic acid comprising a nucleotide sequence encoding a heat shock protein fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said nucleotide sequence being operably linked to a promoter, under conditions such that the heat shock protein peptide-binding fragment is expressed by the cancer cells and associates with peptides of the cell; b) recovering complexes of the heat shock protein fragments noncovalently associated with peptides from the cancer cell; c) administering to the subject an amount of the recovered complexes effective to treat or prevent cancer. In another embodiment, the method further comprises prior to step (a) the step of obtaining cancer cells from the subject and transforming the cancer cells with the nucleic acid. In yet another embodiment, the method further comprises prior to step (a) the step of obtaining cancer cells from one or more individuals and transforming the cancer cells with the nucleic acid, said one or more individuals being different from the subject and having the same type of cancer as the subject.

In various embodiments of the invention such methods, specific Hsp70 family protein peptide-binding fragments are utilized with such methods. In a specific embodiment, the peptide-binding fragment is a fragment of 100 to 200 contiguous amino acids, comprising amino acid residues from the following positions of the amino acid sequence as shown in FIG. 1B (SEQ ID NO:2): 413 to 638, or 424 to 464. In another specific embodiment, the peptide-binding fragment is a fragment of 100 to 200 contiguous amino acids, comprising amino acid residues from position 391 to 615, or from position 400 to 440 of the amino acid sequence as shown in FIG. 1C (SEQ ID NO:3). In yet another embodiment, the heat shock protein is a human hsc70 peptide-binding fragment comprising 100 to 200 contiguous amino acids from positions 391 to 615, or from position 406 to 443 of the amino acid sequence as shown in FIG. 1D (SEQ ID NO:4).

In various embodiments of the invention such methods, specific Hsp90 family protein peptide-binding fragments are utilized with such methods. In a specific embodiment, the peptide-binding fragment is a fragment of 100 to 200 contiguous amino acids, comprising amino acid residues from position 5 to 232 of the amino acid sequence as shown in FIG. 2A (SEQ ID NO:5). In another embodiment, a human hsp84 peptide-binding fragment comprising 100 to 200 contiguous amino acids of positions 5 to 232 of the amino acid sequence as shown in FIG. 2B (SEQ ID NO:6) is utilized. In another embodiment, a gp96 peptide-binding fragment comprising 100 to 200 contiguous amino acids from position 5 to 232 of the amino acid sequence as shown in FIG. 2C (SEQ ID NO:7) is utilized. In yet another embodiment, a gp96 peptide-binding fragment comprising 100 to 200 contiguous amino acids from position 615 to 658 or 624 to 630 of the amino acid sequence as shown in FIG. 2C (SEQ ID NO:7) is utilized. In yet another embodiment, a human PDI protein peptide-binding fragment comprising 100 to 200 contiguous amino acids from position 5 to 232 of the amino acid sequence as shown in FIG. 3 (SEQ ID NO:8) is utilized.

In yet other embodiments, human PDI protein peptide-binding fragments are utilized with the methods of the present invention. In a specific embodiment, a peptide-binding fragment comprising 75 to 139 contiguous amino acids from position 213 to 351 of the amino acid sequence as shown in FIG. 3 (SEQ ID NO:8) is used. In yet another embodiment, a human PDI protein peptide-binding fragment comprising 140 to 298 contiguous amino acids from position 204 to 491 of the amino acid sequence as shown in FIG. 3 (SEQ ID NO:8) is used.

### 4. BRIEF DESCRIPTION OF THE DRAWINGS

FIG. 1A-D. Amino acid sequence of human Hsp70 family proteins. Amino acid residues comprising the entire peptide-binding domain are underscored, amino acids of the P-helix motif are shown in bold, and amino acid residues of the central peptide-binding core are double underscored. A. Amino acid sequence of E. coli DnaK (SEQ ID NO: 1). B. Amino acid sequence of the human Hsp70 family homologue, hBiP, also called grp78 (SEQ ID NO:2). C. Amino acid sequence of the inducible human Hsp70 family homologue, hsp70 (SEQ ID NO:3). D. Amino acid sequence of the constitutive human Hsp70 family homologue, hsc70 (SEQ ID NO:4).

FIG. 2A-C. Amino acid sequences of human Hsp90 family proteins. Amino acid residues comprising the peptide-binding domain are underscored. A. Amino acid sequence of human Hsp 90 family homologue, hsp86, also called hsp90 (SEQ ID NO:5). B. Amino acid sequence of the human Hsp90 family homologue, hsp84, also called hsp90 (SEQ ID NO:6). C. Amino acid sequence of the inducible human Hsp90 family homologue gp96 (SEQ ID NO:7), showing two alternative peptide-binding domains. The amino acid residues of the central peptide-binding core of one of these peptide-binding domains are double underscored.

FIG. 3. Amino acid sequence of human PDI (SEQ ID NO: 8).

### 5. DETAILED DESCRIPTION OF THE INVENTION

The present invention relates to compositions comprising noncovalent complexes of peptide-binding fragments of HSPs with antigenic molecules, and methods for their use as immunotherapeutic agents. The invention is based, in part, on the discovery by the inventors that a fragment containing the portion of an HSP molecule that binds peptides may be used to form complexes with antigenic peptides. Such peptide-binding HPBFs can be used in noncovalent association with antigenic molecules as immunotherapeutic agents. Such HSP-antigenic peptide complexes can be used in immunotherapy, for example, to treat or prevent an infectious disease or cancer.

The terms “HPBF” (HSP peptide-binding fragment), “HSP peptide-binding fragment”, and “peptide-binding HSP fragment” are used interchangeably herein to refer to a polypeptide that comprises an HSP domain that is capable of becoming noncovalently associated with a peptide to form a complex, but that is not a full-length HSP. An HPBF of the invention is capable of eliciting an immune response, and comprises a peptide-binding domain that is contiguous on its N-terminal side with a variable number of amino acids that naturally flank the peptide-binding domain on the N-terminal side (herein termed “x” number of amino acids), and that is contiguous on the C-terminal side with a variable number of amino acids that naturally flank the peptide-binding domain on the C-terminal side (herein termed “y” number of amino acids), wherein x plus y is not more than 400 amino acids. As used herein, a number of “contiguous amino acids that naturally flank the peptide binding domain” refers to a number of contiguous amino acid sequences, adjacent to the peptide-binding domain, in either or both directions, which are also present in the native heat shock protein. Preferably, said fragment lacks one or more other heat shock protein domains.

Such an HPBF may be a polypeptide of less than 100 amino acids, or of 100 to 200 amino acids, 200 to 400 amino acids, or 400 to 800 amino acids. In the preferred embodiment, an HSP peptide-binding fragment is a polypeptide of approximately 100 to 200 amino acids. In various embodiments, x plus y is not more than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 30, 380, 390, or 400 amino acids in length.

In one embodiment, for example, x is not more than 10, 12, 15, 20, 25, 30, 35, 40, 45, 50, 55, 60, 65, 70, 75, 80, 85, 90, 95, 100, 110, 120, 130, 140, 150,160, 170, 180, 190, 200, 210, 220, 230, 240, 250, 260, 270, 280, 290, 300, 310, 320, 330, 340, 350, 360, 30, 380, 390, or 400 amino acids in length. For example, x can be between 0 to 5 amino acids, 5 to 10 amino acids, 10 to 30 amino acids, 30 to 50 amino acids, 50 to 100 amino acids, 100 to 150 amino acids, 150 to 200 amino acids, 200 to 250 amino acids, 250 to 300 amino acids, 300 to 350 amino acids, or 350 to 400 amino acids in length.

In one embodiment, for example, y is not more than 10, 12, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 30, 380, 390, or 400 amino acids in length. For example, y can be between 0 to 5 amino acids, 5 to 10 amino acids, 10 to 30 amino acids, 30 to 50 amino acids, 50 to 100 amino acids, 100 to 150 amino acids, 150 to 200 amino acids, 200 to 250 amino acids, 250 to 300 amino acids, 300 to 350 amino acids, or 350 to 400 amino acids in length.

Non-limiting examples of other heat shock protein domains, one or more of which can be lacking in an HPBF, include: nucleotide-binding domains, such as an ATP-binding domain; domains comprising amino acids involved in ATP hydrolysis; oligomerization domains, such as a dimerization domain; domains encompassing chaperone binding sites; charged domains; middle domains; domains encompassing co-chaperone or cofactor binding sites, such as a domain involved in binding of HSP cofactors having tetratricopeptide repeat domains; hinged domains; linker domains; nuclear localization region; cytoplasmic localization region; and domains involved in protein-folding, provided that the lacking domain is not also required for binding peptide.

In addition, the polypeptide may comprise one or more other domains, and optionally lack all other sequences of the HSP from which the peptide-binding domain is derived. The additional domains of such chimeric HPBFs may be derived from another portion of an HSP molecule, or alternatively, from a heterologous protein. The heterologous protein may be a different heat shock protein or another type of heterologous protein. For example, chimeric HPBF molecules may comprise protein domains that assist in the expression, detection, or recovery of the chimeric HPBF, or in the induction of an immune response.

The invention comprises pharmaceutical compositions comprising HSP peptide-binding fragments in noncovalently bound complexes with antigenic molecules for use in eliciting an immune response for treatment of infectious diseases and cancer. Heat shock proteins useful for obtaining the peptide-binding HSP fragments of the present invention can be selected from among cellular proteins that satisfy any one of the following criteria: the intracellular concentration of such protein increases when a cell is exposed to a stressful stimulus; such protein can bind other proteins or peptides, and can release the bound proteins or peptides in the presence of adenosine triphosphate (ATP) or low pH; or such protein possesses at least 35% homology with any cellular protein having any of the above properties.

HPBFs and HPBF-peptide complexes comprising members of the five major families of HSPs, namely Hsp60, Hsp70, Hsp90, Hsp100, and sHsps, can be prepared by the practice of the present invention. HPBFs include, but are not limited to, the peptide-binding domain fragments of an Hsp70 family protein, such as hsp71, hsc70, BiP, and DnaK, peptide-binding domain fragments of an Hsp90 family protein, such as hsp90, hsp80, hsp81, hsp82, hsp83, hsp84, hsp85, hsp86, hsp100, frp94, endoplasmin, and gp96. In addition, calreticulin, PDI and other proteins in the endoplasmic reticulum that contain thioredoxin-like domain(s), such as but not limited to ERp72 and ERp61, are also encompassed. In various embodiments, the methods provided herein for treatment and prevention of infectious disease and cancer utilize HPBFs comprising a peptide-binding fragment of one type of HSP, or a mixture of peptide-binding fragments of two or more different HSP proteins.

HPBFs may also comprise other related members of the Hsp60, Hsp7, Hsp90, Hsp100, sHsps, calreticulin and PDI families, proeins that are related to HSPs in sequence, for example, having greater than 35% amino acid identity, but whose expression levels are not altered by stress. Therefore it is contemplated that the definition of heat shock or stress protein, as used herein, embraces other proteins, mutants, analogs, and variants thereof having at least 35% to 55%, preferably 55% to 75%, and most preferably 75% to 85% amino acid identity with members of these families whose expression levels in a cell are enhanced in response to a stressful stimulus.

The invention further comprises recombinant cells that express HPBF-peptide complexes. In a preferred embodiment, the HPBFs and that can be prepared by the present invention include peptide-binding fragments of Hsp70, Hsp90, gp96, protein di sulfide isomerase (PDI), calreticulin, or sHSP, alone or in combination. Preferably, the HSPs are human HSPs. A preferred complex comprises a human fragment comprising the peptide-binding domain of Hsp60, Hsp70, Hsp90, calreticulin, PDI, or sHSP, noncovalently bound to a human protein antigen. Using the defined amino acid or cDNA sequences of peptide-binding domains of a given HSP one can make a genetic construct which is transfected into and expressed in a host cell. The genetically engineered host cells may contain one or more copies of a nucleic acid sequence comprising a sequence that encodes an HSP peptide-binding domain, operably associated with regulatory region(s) that drives the expression of the HSP nucleic acid sequence in the host cell. Any nucleic acid sequence encoding an HSP peptide-binding domain, including cDNA and genomic DNA, can be used to prepare the peptide-binding fragments of the invention. It is preferred that the recombinant HSP produced in the host cell or library cell is of the same species as the D intended recipient of the immunogenic composition. Recombinant human HSP is most preferred.

In various embodiments, the invention provides methods for the use of HSP peptide-binding fragments in immunotherapy. An effective amount of complex of an HSP peptide-binding fragment bound to an antigenic molecule can be administered to a patient to elicit an immune response. The HSP peptide-binding fragments can further be used in adoptive immunotherapy treatment. Compositions of complexes of peptide-binding domain fragments of HSPs, noncovalently bound to antigenic molecules, alone or in combination with each other, are administered in combination with the administration of antigen presenting cells sensitized with an HSP-antigenic molecule noncovalent complex to augment the immune response to genotoxic and nongenotoxic factors, tumors and infectious agents. Such HPBF complexes are delivered in combination with immune cells or in combination with interferons, cytokines, etc.

5.1 HSP Peptide-Binding Domain Fragments

Heat shock/stress proteins are composed of structural domains that correspond to their various functional activities, including peptide binding and ATP binding and hydrolysis. In the Hsp70 and Hsp90 families, an ATP hydrolysis cycle regulates substrate binding. Fragments of HSPs lacking the ATP-binding domain can bind peptide substrate; the hydrolysis of ATP is required for release of the peptide and uptake of another substrate. Thus, a fragment of an HSP molecule can be isolated that lacks the ATP-binding domain yet retains the peptide-binding activity of the full-length molecule, and such an HPBF can be used in immunotherapeutic treatments against infectious disease and cancer. The peptide-binding domains of several HSPs of different families are described in detail below.

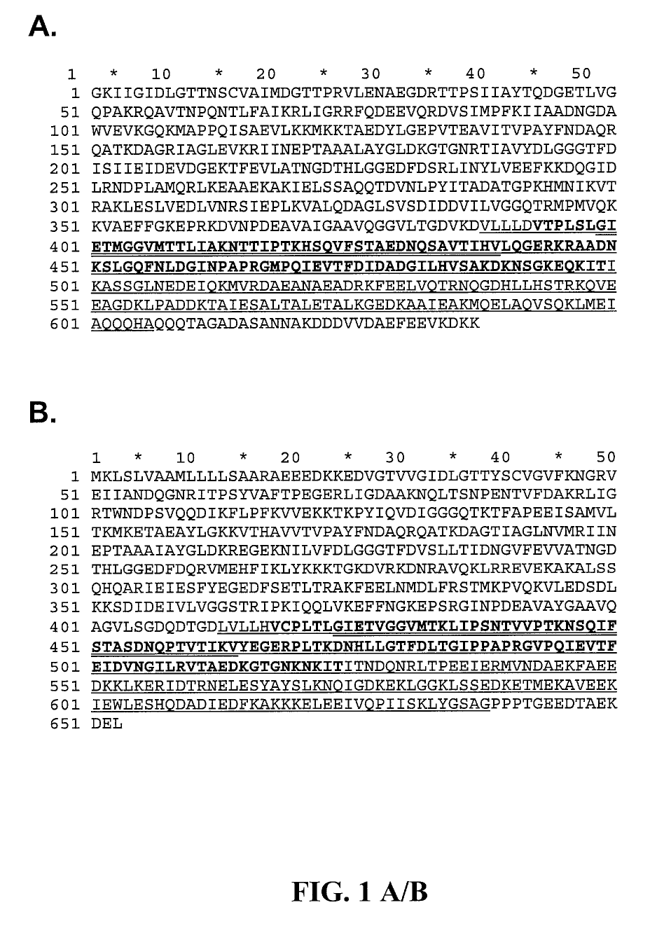

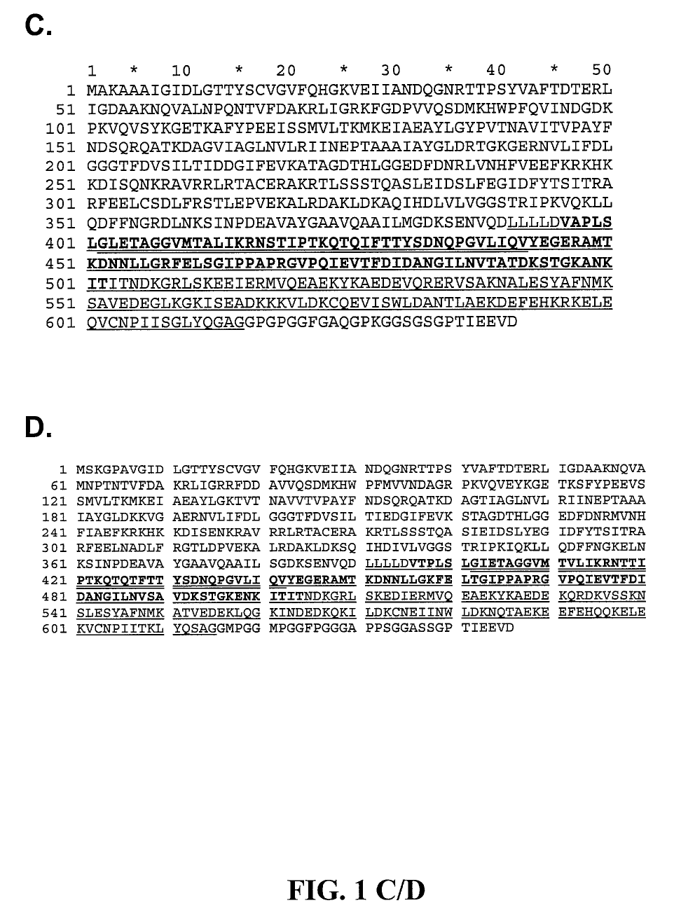

The peptide-binding domain of DnaK, the bacterial homologue of mammalian Hsp70, has been determined (Zhu et al., 1996, Science 272:1606-1614). The peptide-binding domain, spanning amino-acid residues 388 to 606 of DnaK (SEQ ID NO: 1), shown underlined in FIG. 1A, is composed of a β sandwich structure, and an adjacent extended structure of α helices, having overall dimensions of ˜60 by 40 by 15 Å. The β-helix structure, comprising residues 392 to 501, is arranged in two sheets with 4 antiparallel β strands. Substrate binding is almost completely determined by substrate interactions of five amino acid residues at the core of the β-helix structure. This region is exceptionally well conserved in other members of the Hsp70 family, including human Hsc70 and human BiP, where the range of amino acid identity is 65 to 76 percent. The interaction of peptide and the P-helix motif is stabilized by a less well conserved neighboring a-helical region comprising residues 537 to 606.

In accordance with the present invention, HPBFs are constructed or prepared comprising amino acid residues of Hsp70 family peptide-binding domains. In one embodiment, for example, an HPBF comprises a fragment of about 100 contiguous amino acids of positions 388 to 606 of DnaK (SEQ ID NO:1), i.e., the amino acids underscored in FIG. 1A. Preferably, in another embodiment, such an HPBF comprises a fragment of about 100 contiguous amino acids of positions 393 to 500 of DnaK (SEQ ID NO:1), i.e., the amino acids shown in bold in FIG. 1A. More preferably, in yet another embodiment, such an HPBF comprises the central core of the peptide-binding domain a fragment, about 50 to 100 contiguous amino acids of positions 399 to 439 of DnaK (SEQ ID NO:1), i.e., the amino acids double-underscored in FIG 1A.

HPBFs comprising peptide-binding domains of human Hsp70 proteins that correspond to the DnaK peptide-binding domain are also encompassed by the present invention. For example, human Bip, otherwise known as grp78, is a human homologue of DnaK and a member of the Hsp70 family, the amino acid sequence of which is shown in FIG. 1B. In one embodiment, an HPBF comprises a fragment of about 50 to 100 contiguous amino acids of positions 413 to 638 of human BiP (SEQ ID NO:2), i.e., the amino acids underscored in FIG 1B. In a preferred embodiment, such an HPBF comprises a fragment of about 100 contiguous amino acids of positions 418 to 524 of human BiP (SEQ ID NO:2), i.e., the amino acids shown in bold in FIG 1B. In yet a more preferred embodiment, an HPBF comprises about 50 to 100 contiguous amino acids of positions 424 to 464 of human BiP (SEQ ID NO:2), i.e., the amino acids double-underscored in FIG. 1B.

In another embodiment, an HPBF comprises a fragment of about 50 to 100 contiguous amino acids of positions 391 to 615 of the inducible form of a human Hsp70 family protein, hsp71 (SEQ ID NO:3), i.e., the amino acids underscored in FIG. 1C. In a preferred embodiment, such an HPBF comprises a fragment of about 100 contiguous amino acids of positions 395 to 502 of human hsp71 (SEQ ID NO:3), i.e., the amino acids shown in bold in FIG IC. In a more preferred embodiment, an HPBF comprises about 50 to 100 contiguous amino acids of positions 400 to 440 of human hsp71 (SEQ ID NO:3), i.e., the amino acids double-underscored in FIG.1C.

In another embodiment, an HPBF comprises a fragment of about 50 to 100 contiguous amino acids of positions 391 to 615 of the constitutive form of a human Hsp70 family protein, hsc70 (SEQ ID NO:4), i.e., the amino acids underscored in FIG ID. In a preferred embodiment, such an HPBF comprises a fragment of about 100 contiguous amino acids of positions 395 to 504 of human hsp71 (SEQ ID NO:4), i.e., the amino acids shown in bold in FIG 1D. In a more preferred embodiment, an HPBF comprises about 50 to 100 contiguous amino acids of positions 406 to 443 of human hsp71 (SEQ ID NO:4), i.e., the amino acids double-underscored in FIG ID.

In another embodiment, the Hsp70 fragment comprises a peptide-binding domain plus any other domain of interest, such as the ATP-binding domain. In addition, the invention encompasses the corresponding regions of other members of the highly conserved Hsp70 family of proteins. In addition, the invention encompasses chimeric proteins composed of Hsp70 peptide-binding domains linked to other proteins, or fragments or domains thereof. The Hsp90 family of proteins are ubiquitous molecular chaperones which, in eukaryotes, are involved in protein folding of a broad range of important substrates, such as, for example, cellular proteins such as transcription factors, hormone receptors, tyrosine kinases, and anti-oncogenes. Hsp90s are dimeric proteins, having monomers of 80-90 kDa. Members of the Hsp 90 family are highly conserved, especially in their N-terminal and C-terminal regions, which constitute distinct functional domains (Scheibel and Buchner, 1997, in “Guidebook to Chaperones”, Gething, M. J. (ed.), Oxford Univ. Press, Oxford, pp. 147-150). The N-terminal residues 5 to 232 contain a compact ligand-binding pocket comprising the peptide-binding domain, in addition to a nucleotide binding site, a geldanomycin binding site, and an interaction site for unfolded proteins (Stebbins et al., 1997, 89: 239-250; Prodromou et al., 1997, Nat. Struct. Biol. 4: 477-482; Prodromou et al., 1997, Cell 90: 65-75; Wang et al., 1993, J. Biol. Chem. 268: 26049-51). Adjacent to the N-terminal ligand-binding pocket, from about residues 210 to 272, is a highly charged linker domain, which modulates the activity of the N-terminal domain, by decreasing the affinity of peptide-bound N-terminal domain for binding of ATP and geldanamycin (Scheibel et al., 1998, Proc. Natl. Acad. Sci. USA 95: 1495-1499). The C-terminal domain comprises the oligomerization domain (residues 676 to 719; Wearsch and Nicchitta, 1996, Biochemistry 35:16760-9), an ATP-independent chaperone site (residues 262 to 709; Scheibel et al., 1998, Proc. Natl. Acad. Sci. USA 95: 1495-1499), and a TPR domain, which specifically binds Hsp90 cofactors having TPR (tetratricopeptide repeat) domains (residues 558 to 724; Young et al., 1998, J. Biol. Chem. 273:18007-10; Young et al., 1997, FEBS Lett. 418: 139-143; Carrello et al., 1999, J. Biol. Chem., 274: 2682-9). The N-terminal and C-terminal domains are connected by a large middle domain, from about residues 294 to 600, which is highly conserved among Hsp90 family proteins. This region comprises a region involved in nuclear localization between amino acid residues 381 and 581, a cytoplasmic localization region between residues 287 and 728, and a glucocorticosteroid receptor interaction domain between residues 446 and 581 (Jibard et al., 1999, Exp. Cell. Res., 15: 461-74). An alternative peptide-binding site of the Hsp90 family member, gp96, has been mapped to amino acid residues 615 through 658 of gp96, with a central peptide-binding core mapping to amino acid residues 624 through 630 of gp96 (Linderoth et. al., 2000, J. Biol. Chem. 275: 5472-77).

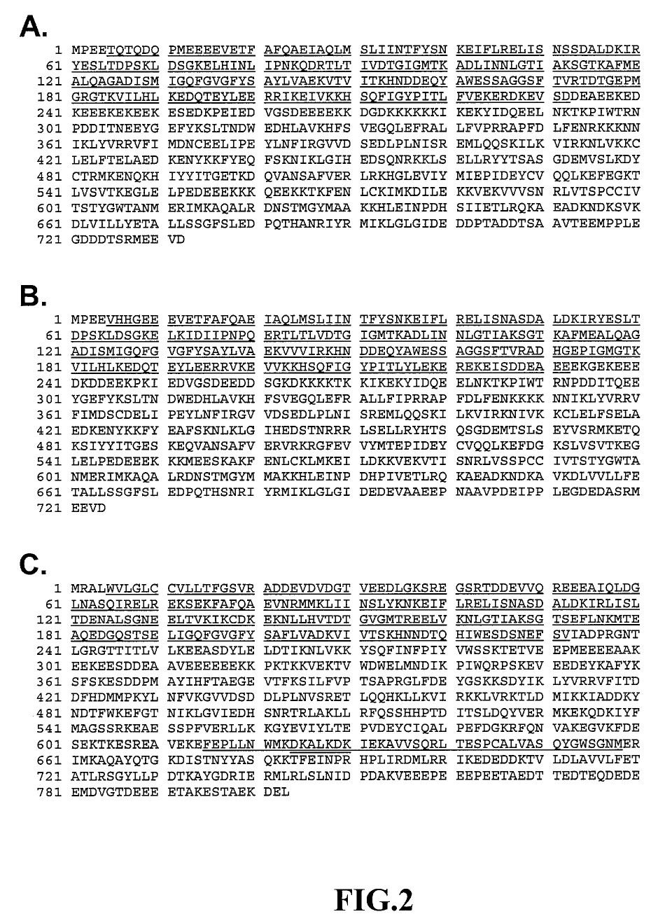

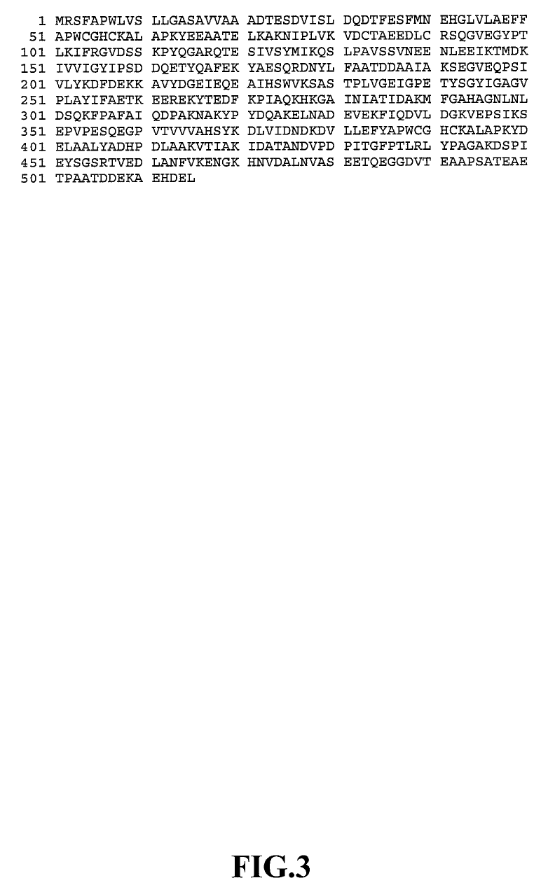

Hsp90 peptide-binding fragments can be constructed or prepared that encompass the ligand domain, from about amino acid residues 5 to 272, or a peptide-binding fragment thereof. In another embodiment, an Hsp90 peptide-binding fragment can comprise amino acids 5 to 232, or a peptide-binding fragment thereof. In another embodiment, gp96 peptide-binding fragments can be constructed or prepared that encompasses amino acid residues 615 through 658 of gp96, or a peptide-binding fragment thereof. In another embodiment, an gp96 peptide-binding fragment can comprise amino acids 624 through 630 of gp96, or a peptide-binding fragment thereof (shown by the doubly underscored amino acids in FIG. 2C [SEQ ID NO:7]).

Peptide-binding fragments from members of the Hsp90 family used in accordance with the invention may include the substrate-binding domain either alone or together with additional domains, such as, for example, a second substrate-binding domain, the oligomerization domain, the C-terminal domain, the charged domain, the chaperone site, or the substrate-binding domain together with any other domain of interest, such as the charged domain or the nucleotide-binding site (but optionally not containing other parts of the molecule). In addition, the invention encompasses the corresponding regions of other members of the highly conserved Hsp90 family of proteins. In addition, the invention encompasses chimeric proteins composed of Hsp90 peptide-binding domains linked to other proteins, or fragments or domains thereof.

Protein disulfide isomerase (PDI) is an HSP of 491 amino acids, involved in folding of many disulfide-bonded proteins. Structure-function studies using PDI fragments have lead to a definition of PDI functional domains (Klappa et al.,1998, EMBO J., 17:927-935). These studies indicate that PDI contains various domains that contribute to peptide binding, unlike the Hsp90 and Hsp70 families of proteins, which appear to have discrete substrate binding domains. A PDI fragment that includes amino acid residues 213 to 351 is capable of binding short peptides (10-15 amino acids). A larger fragment, including amino acids 204 to 491, is able to bind of larger polypeptides of 25 to 50 amino acids. An even larger PDI fragment, including amino acids residues 1 to 370 is required for more complex substrates, such as larger polypeptides.

In a preferred embodiment, PDI peptide-binding fragments are constructed for use in accordance with the present invention that encompass the minimal peptide-binding domain (amino acids 213 to 351), capable of binding small peptides (10-15 amino acids). In an alternative embodiment, larger PDI peptide-binding fragments can be constructed that encompass the peptide-binding domain (amino acids 204 to 491) capable of binding larger polypeptides (for example, 28 amino acids). In yet another embodiment, a large PDI peptide-binding fragment (amino acids 1 to 370), capable of binding complex polypeptides (such as ‘scrambled’ RNase A, a natural substrate of PDI). Combinations of any of these domains together with each other or with other PDI domains, such as the ATP-binding domain, or combinations of any of these domains together with domains of heterologous proteins, for example other HSP domains, are also within the scope of the invention. An entire domain or a fragment thereof can be used to prepare such constructs.

Small HSPs (sHSPs) range in size from 12K to 42K and form large multimeric structures of 200K to 400K. sHSPs have a wide range of cellular functions, including thermoprotection in vivo and chaperoning in vitro. The crystal structure of an sHSP (Kim et al., 1998, Nature 394:595-599) shows that 24 monomeric subunits of sHSPs form a hollow spherical complex of octahedral symmetry, the N-terminal residues of which seem to be important for chaperone activity. The entire domain encompassing such N-terminal residues, or a fragment thereof, can be used to construct peptide-binding fragments for use in the present invention.

While the precise boundaries of the peptide-binding domain of the above-mentioned HSPs have been determined, others can be determined by sequence comparison to other members of the analogous HSP family. The high conservation of the substrate-binding amino acids and domains within HSP protein families makes it possible to predict the corresponding peptide-binding region in a given heat shock protein family member. Even HSPs whose domain structure have not yet been precisely mapped are within the scope of the present invention. For example, the endoplasmic reticular Hsp90 family protein, gp96, is known to contain several ATP-binding regions and presumably possesses a peptide-binding domain since gp96 is known to bind peptides.

As discussed above, the details of the peptide-binding domains of several HSPs are known. However, it is not necessary to know the precise boundaries of the peptide-binding domain of an HSP to use such an HPBF comprising a peptide-binding domain with the methods of the invention. Recombinant DNA techniques can be readily utilized to generate recombinant fragments of HSP genes, and standard recombinant techniques can be used to express such HSP gene fragments. Such recombinant HPBFs can be assayed for peptide binding activity (see for example, Klappa et al.,1998, EMBO J., 17:927-935) for their ability to elicit an immune response (as described in Section 5.4.1, herein). Such peptide-binding domains can be purified, or used in a non-purified form. Alternatively, HPBFs comprising peptide binding domains can be generated from native HSPs by protease cleavage of native HSPs or native HSP-peptide complexes, as described in detail below in Section 5.2.3.

Alternatively, the peptide-binding domain of an HSP may be predicted from its primary sequence, by sequence comparison to known HSPs and other peptide-binding proteins. HSPs share similar structural properties with peptide-binding domains from other peptide-binding proteins, such as class I and class II major histocampatibility (MHC) complex antigen presenting molecules, SH2 and SH3 domains, protein kinases and phosphatase, proteases, antibodies, in that they bind peptide segments in extended conformations. Thus, one can search protein and nucleotide sequence databases to identify sequences with various degrees of similarities to these conserved peptide-binding motifs, using search programs such as FASTA and BLAST which rank the similar sequences by alignment scores and statistics.

To determine a region of identity between two amino acid sequences or nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions x 100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

Functionally active derivatives and analogs of HSP peptide-binding domains are also within the scope of the present invention. In one embodiment, for example, Hsp70 peptide-binding domain derivatives and analogs can be designed. By computer modeling the three dimensional structure of the Hsp70 peptide-binding site, Hsp70 variants can be designed in which amino acid residues not involved in peptide binding or structurally important determinants may be substituted for the wild-type residues.

In another embodiment, where the three dimensional structure of the peptide-binding site of an HSP is unknown, its structure can be determined. This can be done using methods known to those of skill in the art, including X-ray crystallography. Alternatively, solid or liquid phase NMR can be used to determine certain intra-molecular distances. Any other experimental method of structure determination can be used to obtain partial or complete molecular structures. The molecular structures may be measured with a complexed peptide or other ligand, natural or artificial.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Examples of molecular modeling systems are the CHARMm and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

5.2 Preparation of HSP Peptide-Binding Fragments

The HSP peptide-binding fragment polypeptide of the invention may be produced by recombinant DNA techniques, synthetic methods, or by enzymatic or chemical cleavage of native HSPs. Described herein are methods for producing such HSP peptide-binding fragments.

5.2.1. Production of Recombinant HSP Peptide-Binding Fragments

5.2.1.1 Isolation of HSP Gene Sequences

In various aspects, the invention relates to amino acid sequences of peptide-binding fragments of HSPs. Nucleic acids encoding the HPBFs are provided, as well as nucleic acids complementary to and capable of hybridizing to such nucleic acids.

Any eukaryotic cell may serve as the nucleic acid source for obtaining the coding region of an hsp gene. Nucleic acid sequences encoding HSPs can be isolated from vertebrate, mammalian, as well as primate sources, including humans.

Amino acid sequences and nucleotide sequences of naturally occurring HSPs are generally available in sequence databases, such as GenBank. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics. Such nucleotide sequences of non-limiting examples of HSPs that can be used for preparation of the HSP peptide-binding fragments of the invention are as follows: human Hsp70 Genbank Accession No. NM_{—}005345, Sargent et al., 1989, Proc. Natl. Acad. Sci. U.S.A., 86:1968-1972; human Hsp90, Genbank Accession No. X15183, Yamazaki et al., Nucl. Acids Res. 17:7108; human gp96: Genbank Accession No. X15187, Maki et al., 1990, Proc. Natl. Acad Sci., 87: 5658-5562; human BiP: Genbank Accession No. Ml 9645; Ting et al., 1988, DNA 7: 275-286; human Hsp27, Genbank Accession No. M24743; Hickey et al., 1986, Nucleic Acids Res. 14:4127-45; mouse Hsp70 : Genbank Accession No. M35021, Hunt et al., 1990, Gene, 87:199-204; mouse gp96: Genbank Accession No. M16370, Srivastava et al., 1987, Proc. Natl. Acad. Sci., 85:3807-3811; and mouse BiP: Genbank Accession No. U16277, Haas et al., 1988, Proc. Natl. Acad. Sci. U.S.A., 85: 2250-2254. Due to the degeneracy of the genetic code, the term “HSP gene”, as used herein, refers not only to the naturally occurring nucleotide sequence but also encompasses all the other degenerate DNA sequences that encode the HSP. The DNA may be obtained by standard procedures known in the art by DNA amplification or molecular cloning directly from a tissue, cell culture, or cloned DNA (e.g., a DNA “library”). Clones derived from genomic DNA may contain regulatory and intron DNA regions in addition to coding regions; clones derived from cDNA will contain only exon sequences. Whatever the source, the hsp gene should be cloned into a suitable vector for propagation of the gene.

In a preferred embodiment, DNA can be amplified from genomic or cDNA by polymerase chain reaction (PCR) amplification using primers designed from the known sequence of a related or homologous HSP. PCR is used to amplify the desired sequence in DNA clone or a genomic or cDNA library, prior to selection. PCR can be carried out, e.g., by use of a thermal cycler and Taq polymerase (Gene Amp®). The DNA being amplified can include cDNA or genomic DNA from any species. Oligonucleotide primers representing known nucleic acid sequences of related HSPs can be used as primers in PCR. In a preferred aspect, the oligonucleotide primers represent at least part of the hsp gene that is highly conserved between HSPs of different species. One can choose to synthesize several different degenerate primers, for use in the PCR reactions. It is also possible to vary the stringency of hybridization conditions used in priming the PCR reactions, to allow for greater or lesser degrees of nucleotide sequence similarity between the known HSP nucleotide sequence and the nucleic acid homologue being isolated. For cross species hybridization, low stringency conditions are preferred. For same species hybridization, moderately stringent conditions are preferred. After successful amplification, the sequence encoding an HSP may be cloned and sequenced. If the size of the coding region of the hsp gene being amplified is too large to be amplified in a single PCR, several PCR covering the entire gene, preferably with overlapping regions, may be carried out, and the products of the PCR ligated together to form the entire coding sequence. Alternatively, if a segment of an hsp gene is amplified, that segment may be cloned, and utilized as a probe to isolate a complete cDNA or genomic clone.

In another embodiment, for the molecular cloning of an hsp gene from genomic DNA, DNA fragments are generated to form a genomic library. Since some of the sequences encoding related HSPs are available and can be purified and labeled, the cloned DNA fragments in the genomic DNA library may be screened by nucleic acid hybridization to the labeled probe (Benton and Davis, 1977, Science 196:180; Grunstein and Hogness, 1975, Proc. Natl. Acad. Sci. U.S.A. 72:3961). Those DNA fragments with substantial homology to the probe will hybridize. It is also possible to identify the appropriate fragment by restriction enzyme digestion(s) and comparison of fragment sizes with those expected according to a known restriction map if such is available. Alternatives to isolating the hsp genomic DNA include, but are not limited to, chemically synthesizing the gene sequence itself from a known sequence or making cDNA to the mRNA which encodes the HSP. For example, RNA for cDNA cloning of the hsp gene can be isolated from cells which express the HSP. A cDNA library may be generated by methods known in the art and screened by methods, such as those disclosed for screening a genomic DNA library. If an antibody to the HSP is available, the HSP may be identified by binding of labeled antibody to the putatively HSP synthesizing clones.

Other specific embodiments for the cloning of a nucleotide sequence encoding an HSP, are presented as examples but not by way of limitation, as follows:

In a specific embodiment, nucleotide sequences encoding heat shock protein within a family can be identified and obtained by hybridization with a probe comprising nucleotide sequence encoding an HSP under conditions of low to medium stringency.

By way of example and not limitation, procedures using such conditions of low stringency are as follows (see also Shilo and Weinberg, 1981, Proc. Natl. Acad. Sci. USA 78:6789-6792). Filters containing DNA are pretreated for 6 h at 40° C. in a solution containing 35% formamide, 5X SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20 X 106 cpm ³²P-labeled probe is used. Filters are incubated in hybridization mixture for 18-20 h at 40° C., and then washed for 1.5 h at 55° C. in a solution containing 2X SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

An HSP gene fragment containing the peptide-binding domain can be inserted into an appropriate cloning vector and introduced into host cells so that many copies of the gene sequence are generated. A large number of vector-host systems known in the art may be used such as, but not limited to, bacteriophages such as lambda derivatives, or plasmids such as pBR322 or pUC plasmid derivatives or the Bluescript vector (Stratagene).

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson, C., et al., 1978, J. Biol. Chem 253:6551), oligonucleotide-directed mutagenesis (Smith, 1985, Ann. Rev. Genet. 19:423-463; Hill et al., 1987, Methods Enzymol. 155:558-568), PCR-based overlap extension (Ho et al., 1989, Gene 77:51-59), PCR-based megaprimer mutagenesis (Sarkar et al., 1990, Biotechniques, 8:404-407), etc. Modifications can be confirmed by double stranded dideoxy DNA sequencing.

The polymerase chain reaction (PCR) is commonly used for obtaining genes or gene fragments of interest. For example, a nucleotide sequence encoding an HSP peptide-binding fragment of any desired length can be generated using PCR primers that flank the nucleotide sequence encoding the peptide-binding domain. Alternatively, an hsp gene sequence can be cleaved at appropriate sites with restriction endonuclease(s) if such sites are available, releasing a fragment of DNA encoding the peptide-binding domain. If convenient restriction sites are not available, they may be created in the appropriate positions by site-directed mutagenesis and/or DNA amplification methods known in the art (see, for example, Shankarappa et al., 1992, PCR Method Appl. 1:277-278). The DNA fragment that encodes the HSP peptide-binding domain is then isolated, and ligated into an appropriate expression vector, care being taken to ensure that the proper translation reading frame is maintained.

The HSP peptide-binding fragments of the invention may be expressed as fusion proteins to facilitate recovery and purification from the cells in which they are expressed. For example, the HPBF may contain a signal sequence leader peptide to direct its translocation across the ER membrane for secretion into culture medium. Further, the HPBF may contain an affinity label, such as a affinity label, fused to any portion of the HPBF not involved in binding antigenic peptide, such as for example, the carboxyl terminal.

The affinity label can be used to facilitate purification of the protein, by binding to an affinity partner molecule.

Various methods for production of such fusion proteins are well known in the art. The manipulations which result in their production can occur at the gene or protein level, preferably at the gene level. For example, the cloned coding region of an HPBF may be modified by any of numerous recombinant DNA methods known in the art (Sambrook et al., 1990, Molecular Cloning, A Laboratory Manual, 2d ed., Cold Spring Harbor Laboratory, Cold Spring Harbor, N.Y.; Ausubel et al., in Chapter 8 of Current Protocols in Molecular Biology, Greene Publishing Associates and Wiley Interscience, New York). It will be apparent from the following discussion that substitutions, deletions, insertions, or any combination thereof are introduced or combined to arrive at a final nucleotide sequence encoding an HSP peptide-binding fragment.

In various embodiments, fusion proteins comprising the HSP peptide-binding fragment may be made using recombinant DNA techniques. For example, a recombinant gene encoding an HSP peptide-binding fragment may be constructed by introducing an hsp gene fragment in the proper reading frame into a vector containing the sequence of an affinity label, such that the HPBF is expressed as a peptide-tagged fusion protein. Affinity labels, which may be recognized by specific binding partners, may be used for affinity purification of the HPBF.

In a preferred embodiment, the affinity label is fused at its amino terminal to the carboxyl terminal of the HSP. The precise site at which the fusion is made in the carboxyl terminal is not critical. The optimal site can be determined by routine experimentation. The immunogenicities of the HSP peptide-binding fragment can be tested by methods described in Section 5.4.1.

A variety of affinity labels known in the art may be used, such as, but not limited to, the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels may impart fluorescent properties to an HPBF, e.g., portions of green fluorescent protein and the like. Other possible affinity labels are short amino acid sequences to which monoclonal antibodies are available, such as but not limited to the following well known examples, the FLAG epitope, the myc epitope at amino acids 408-439, the influenza virus hemagglutinin (HA) epitope. Other affinity labels are recognized by specific binding partners and thus facilitate isolation by affinity binding to the binding partner which can be immobilized onto a solid support. Some affinity labels may afford the HPBF novel structural properties, such as the ability to form multimers. Dimerization of an HPBF with a bound peptide may increase avidity of interaction between the HPBF and its partner in the course of antigen presentation. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or portions of the immunoglobulin molecule containing sites for interchain disulfide bonds, could lead to the formation of multimers. As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based affinity label may be obtained from IgG-1, -2, -3, or -4 subtypes, IgA, IgE, IgD, or IgM, but preferably IgG1. Preferably, a human immunoglobulin is used when the HPBF is intended for in vivo use for humans. Many DNA encoding immunoglobulin light or heavy chain constant regions is known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad. Sci. U.S.A., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, the HPBF-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate the HPBF containing the affinity label. In many instances, there is no need to develop specific antibodies to the HPBF.

A particularly preferred embodiment is a fusion of an HSP peptide-binding fragment to the hinge, the CH2 and CH3 domains of human immunoglobulin G- 1 (IgG- 1; see Bowen et al.,1996, J. Immunol. 156:442-49). This hinge region contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of HPBF from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816). Preferred leader sequences for targeting HPBF expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (Maclntyre et al., 1990, Mol. Gen. Genet. 221:466-74; Kaiser et al., 1987, Science, 235:312-317).

DNA sequences encoding a desired affinity label or leader peptide, which may be readily obtained from libraries, produced synthetically, or may be available from commercial suppliers, are suitable for the practice of this invention. Such methods are well known in the art.

5.2.1.2 Recombinant Expression

In various embodiments of the invention, sequences encoding an HPBF are inserted into an expression vector for propagation and expression in recombinant cells. An expression construct, as used herein, refers to a nucleotide sequence encoding an HPBF operably associated with one or more regulatory regions which allows expression of the HPBF in an appropriate host cell. “Operably-associated” refers to an association in which the regulatory regions and the HPBF sequence to be expressed are joined and positioned in such a way as to permit transcription, and ultimately, translation.

Vectors based on E. coli are the most popular and versatile systems for high level expression of foreign proteins (Makrides, 1996, Microbiol Rev, 60:512-538). Non-limiting examples of regulatory regions that can be used for expression in E. coli may include but not limited to lac, trp, lpp, phoA, recA, tac, λP_{L} and phage T3 and T7 promoters (Makrides, 1996, Microbiol Rev, 60:512-538). Non-limiting examples of prokaryotic expression vectors may include the λgt vector series such as λgt11 (Huynh et al., 1984 in “DNA Cloning Techniques”, Vol. 1: A Practical Approach (D. Glover, ed.), pp. 49-78, IRL Press, Oxford), and the pET vector series (Studier et al., 1990, Methods Enzymol., 185:60-89). However, a potential drawback of a prokaryotic host-vector system is the inability to perform many of the post-translational processing events of mammalian cells. Thus, an eukaryotic host-vector system is preferred, a mammalian host-vector system is more preferred, and a human host-vector system is the most preferred.

The regulatory regions necessary for transcription of the HPBF can be provided by the expression vector. A translation initiation codon (ATG) may also be provided to express a nucleotide sequence encoding an HPBF that lacks an initiation codon. In a compatible host-construct system, cellular proteins required for transcription, such as RNA polymerase and transcription factors, will bind to the regulatory regions on the expression construct to effect transcription of the HPBF sequence in the host organism. The precise nature of the regulatory regions needed for gene expression may vary from host cell to host cell. Generally, a promoter is required which is capable of binding RNA polymerase to initiate the transcription of an operably-associated nucleic acid sequence. Such regulatory regions may include those 5′-non-coding sequences involved with initiation of transcription and translation, such as the TATA box, the cap site, a CAAT box, and the like. The non-coding region 3′ to the coding sequence may contain transcriptional termination regulatory sequences, such as terminators and polyadenylation sites.

Both constitutive and inducible regulatory regions may be used for expression of the HPBF. It may be desirable to use inducible promoters when the conditions optimal for growth of the recombinant cells and the conditions for high level expression of the HPBF are different. Examples of useful regulatory regions are provided in the next section below.

For expression of HSP peptide-binding fragments in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LTR), the P-interferon gene, and the Hsp70 gene (Williams et al., 1989, Cancer Res. 49:2735-42 ; Taylor et al., 1990, Mol. Cell Biol., 10:165-75). It may be advantageous to use heat shock promoters or stress promoters to drive expression of the HPBF in recombinant host cells.

The following animal regulatory regions, which exhibit tissue specificity and have been utilized in transgenic animals, can also be used in tumor cells of a particular tissue type: elastase I gene control region which is active in pancreatic acinar cells (Swift et al., 1984, Cell 38:639-646; Omitz et al, 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al, 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha I-antitrypsin gene control region which is active in the liver (Kelsey et al., 1987, Genes and Devel. 1:161-171), beta-globin gene control region which is active in myeloid cells (Mogram et aL, 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

The efficiency of expression of the HPBF in a host cell may be enhanced by the inclusion of appropriate transcription enhancer elements in the expression vector, such as those found in SV40 virus, Hepatitis B virus, cytomegalovirus, immunoglobulin genes, metallothionein, β-actin (see Bittner et al., 1987, Methods in Enzymol. 153:516-544; Gorman, 1990, Curr. Op. in Biotechnol. 1:36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA.

It may also be advantageous to use shuttle vectors that can be replicated and maintained in at least two types of host cells.

In addition, the expression vector may contain selectable or screenable marker genes for initially isolating or identifying host cells that contain DNA encoding an HPBF. For long term, high yield production of HPBF-peptide complexes, stable expression in mammalian cells is preferred. A number of selection systems may be used for mammalian cells, including, but not limited, to the Herpes simplex virus thymidine kinase (Wigler et al., 1977, Cell 11:223), hypoxanthine-guanine phosphoribosyltransferase (Szybalski and Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dihydrofolate reductase (dhfr), which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neomycin phosphotransferase (neo), which confers resistance to the aminoglycoside G-418 (Colberre-Garapin et al., 1981, J. Mol. Biol. 150:1); and hygromycin phosphotransferase (hyg), which confers resistance to hygromycin (Santerre et al., 1984, Gene 30:147). Other selectable markers, such as but not limited to histidinol and Zeocin™ can also be used.

In order to insert the HPBF DNA sequence into the cloning site of a vector, DNA sequences with regulatory functions, such as promoters, must be attached to DNA sequences encoding the HSP peptide-binding region. To do this, linkers or adapters providing the appropriate compatible restriction sites may be ligated to the ends of cDNA or synthetic DNA encoding an HPBF, by techniques well known in the art (Wu et al., 1987, Methods in Enzymol 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising an HPBF sequence operably associated with regulatory regions can be directly introduced into appropriate host cells for expression and production of HPBF-peptide complexes without further cloning (see, for example, U.S. Pat. No. 5,580,859). The expression constructs may also contain DNA sequences that facilitate integration of the HPBF sequence into the genome of the host cell, e.g., via homologous recombination. In this instance, it is not necessary to employ an expression vector comprising a replication origin suitable for appropriate host cells in order to propagate and express the HPBF in the host cells.

Expression constructs containing cloned nucleotide sequence encoding HSP peptide-binding fragments can be introduced into the host cell by a variety of techniques known in the art, including but not limited to, for prokaryotic cells, bacterial transformation (Hanahan, 1985, in DNA Cloning, A Practical Approach, 1:109-136), and for eukaryotic cells, calcium phosphate mediated transfection (Wigler et al., 1977, Cell 11:223-232), liposome-mediated transfection (Schaefer-Ridder et al., 1982, Science 215:166-168), electroporation (Wolff et al, 1987, Proc Natl Acad Sci 84:3344), and microinjection (Cappechi, 1980, Cell 22:479-488). Co-expression of an HPBF and an antigen in the same host cell can be achieved by essentially the same methods.

For long term, high yield production of properly processed HPBFs or HPBF-peptide complexes, stable expression in mammalian cells is preferred. Cell lines that stably express HPBFs or HPBF-peptide complexes may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while HPBF is expressed continuously.

The recombinant cells may be cultured under standard conditions of temperature, incubation time, optical density, and media composition. Alternatively, a recombinant antigenic cells may be cultured under conditions emulating the nutritional and physiological requirements of the cancer cell or infected cell. However, conditions for growth of recombinant cells may be different from those for expression of HPBFs and antigenic proteins. Modified culture conditions and media may also be used to enhance production of HSP-peptide complexes. For example, recombinant cells containing HPBFs with their cognate promoters may be exposed to heat or other environmental stress, or chemical stress. Any techniques known in the art may be applied to establish the optimal conditions for producing HPBF or HPBF-peptide complexes.

5.2.1.3 Purification Methods for Recombinant HSP Peptide-Binding Fragments

Generally, the HSP peptide-binding fragments of the invention can be recovered and purified from recombinant cell cultures by known methods, including ammonium sulfate precipitation, acid extraction, anion or cation exchange chromatography, phosphocellulose chromatography, immunoaffinity chromatography, hydroxyapatite chromatography, and lectin chromatography.

The invention provides methods for purification of recombinant HPBFs by affinity purification, based on the properties of the affinity label present on the HSP peptide-binding fragment. One approach is based on specific molecular interactions between a tag and its binding partner. The other approach relies on the immunospecific binding of an antibody to an epitope present on the tag. The principle of affinity chromatography well known in the art is generally applicable to both of these approaches.

Described below are several methods based on specific molecular interactions of a tag and its binding partner.

A method that is generally applicable to purifying recombinant HSPs that are fused to the constant regions of immunoglobulin is protein A affinity chromatography, a technique that is well known in the art. Staphylococcus protein A is a 42 kD polypeptide that binds specifically to a region located between the second and third constant regions of heavy chain immunoglobulins. Because of the Fc domains of different classes, subclasses and species of immunoglobulins, affinity of protein A for human Fc regions is strong, but may vary with other species. Subclasses that are less preferred include human IgG-3, and most rat subclasses. For certain subclasses, protein G (of Streptococci) may be used in place of protein A in the purification. Protein-A sepharose (Pharmacia or Biorad) is a commonly used solid phase for affinity purification of antibodies, and can be used essentially in the same manner for the purification of HPBF fused to an immunoglobulin Fc fragment. Secreted HPBF present in cell supernatant binds specifically to protein A on the solid phase, while the contaminants are washed away. Bound HPBF can be eluted by various buffer systems known in the art, including a succession of citrate, acetate and glycine-HCl buffers which gradually lowers the pH. This method is less preferred if the recombinant cells also produce antibodies which will be copurified with the HPBF. See, for example, Langone, 1982, J. Immunol. meth. 51:3; Wilchek et al., 1982, Biochem. Intl. 4:629; Sjobring et al., 1991, J. Biol. Chem. 26:399; page 617-618, in Antibodies A Laboratory Manual, edited by Harlow and Lane, Cold Spring Harbor laboratory, 1988.

Alternatively, a polyhistidine tag may be used, in which case, the HSP peptide-binding fragment can be purified by metal chelate chromatography. The polyhistidine tag, usually a sequence of six histidines, has a high affinity for divalent metal ions, such as nickel ions (Ni²⁺), which can be immobilized on a solid phase, such as nitrilotriacetic acid matrices. Polyhistidine has a well characterized affinity for Ni²⁺-NTA-agarose, and can be eluted with either of two mild treatments: imidazole (0.1 -0.2 M) will effectively compete with the resin for binding sites; or lowering the pH just below 6.0 will protonate the histidine side-chains and disrupt the binding. The purification method comprises loading the cell culture supernatant onto the Ni²⁺-NTA-agarose column, washing the contaminants through, and eluting the HPBF with imidazole or weak acid. Ni²⁺-NTA-agarose can be obtained from commercial suppliers such as Sigma (St. Louis) and Qiagen. Antibodies that recognize the polyhistidine tag are also available which can be used to detect and quantify the HPBF.

Another exemplary affinity label that can be used is the glutathione-S-transferase (GST) sequence, originally cloned from the helminth, Schistosoma japonicum. In general, an HSP peptide-binding fragment-GST fusion expressed in a prokaryotic host cell, such as E. coli, can be purified from the cell culture supernatant by absorption with glutathione agarose beads, followed by elution in the presence of free reduced glutathione at neutral pH. Denaturing conditions are not required at any stage during purification, and therefore, it may be desirable for use in the loading of immobilized HSP peptide-binding fragments with antigenic peptides. Moreover, since GST is known to form dimers under certain conditions, dimeric HPBFs may be obtained. See, Smith, 1993, Methods Mol. Cell Bio. 4:220-229.

Another useful affinity label that can be used is the maltose binding protein (MBP) of E. coli, which is encoded by the malE gene. The secreted HPBF-MBP present in the cell supernatant binds to amylose resin while contaminants are washed away. The bound HPBF-MBP is eluted from the amylose resin by maltose. See, for example, Guan et al., 1987, Gene 67:21-30.

The second approach for purifying HSP peptide-binding fragments is applicable to affinity labels that contain an epitope for which polyclonal or monoclonal antibodies are available. Various methods known in the art for purification of protein by immunospecific binding, such as immunoaffinity chromatography, and immunoprecipitation, can be used. See, for example, Chapter 13 in “Antibodies A Laboratory Manual”, 1988, Harlow and Lane, (eds.), Cold Spring Harbor Laboratory, N.Y. and Chapter 8, Sections I and II, in “Current Protocols in Immunology”, 1991, Coligan et al. (eds.), John Wiley,; the disclosure of which are both incorporated by reference herein.

The embodiments described above may be used to recover and purify HPBF-peptide complexes from the cell culture medium of mammalian cells, such as human cells expressing an HSP peptide-binding fragment of the invention. The methods can be adapted to perform medium and large scale purification of an HSP peptide-binding fragment and/or fragment-peptide complexes. Methods that do not require lowering pH or denaturing conditions are most preferred for purification of HPBF-peptide complexes. The methods may be used to isolate HSP peptide-binding fragments from eukaryotic cells, for example, cancer cells, tissues, isolated cells, or immortalized eukaryote cell lines infected with an intracellular pathogen, or cells obtained from a subject infected with a pathogen.

5.2.1.4 Host-Vector Systems

Described herein are systems of vectors and host cells that can be used for the expression of HSP peptide-binding fragments. A variety of expression vectors may be used in the present invention which include, but are not limited to, plasmids, cosmids, phage, phagemids, or modified viruses. Typically, such expression vectors comprise a functional origin of replication for propagation of the vector in an appropriate host cell, one or more restriction endonuclease sites for insertion of the HPBF gene sequence, and one or more selection markers. The expression vector must be used with a compatible host cell which may be derived from a prokaryotic or an eukaryotic organism including but not limited to bacteria, yeasts, insects, mammals, and humans.

Expression constructs and vectors are introduced into host cells for the purpose of producing an HSP peptide-binding fragment. Any cell type that can produce HSPs and is compatible with the expression vector may be used, including those that have been cultured in vitro or genetically engineered. Host cells may be obtained from normal or affected subjects, including healthy humans, cancer patients, and patients with an infectious disease, private laboratory deposits, public culture collections such as the American Type Culture Collection, or from commercial suppliers.

Different host cells have characteristic and specific mechanisms for the post-translational processing and modification of proteins. A host cell may be chosen which modifies and processes the expressed gene products in a specific fashion similar to the way the recipient processes HSPs. For the purpose of producing large amounts of HSP, it is preferable that the type of host cell used in the present invention has been used for expression of heterologous genes, and is reasonably well characterized and developed for large-scale production processes. In a specific embodiment, the host cells are from the same patient to whom HPBF-peptide complexes or recombinant cells expressing HPBF-peptide complexes are going to be administered. Otherwise said, the cells used to express the HPBF and used subsequently to administer immunotherapy to a subject are autologous to the subject.

Preferred mammalian host cells include but are not limited to those derived from humans, monkeys and rodents, (see, for example, Kriegler M. in “Gene Transfer and Expression: A Laboratory Manual”, New York, Freeman & Co. 1990), such as monkey kidney cell line transformed by SV40 (COS-7, ATCC CRL 1651); human embryonic kidney line (293, 293-EBNA, or 293 cells subcloned for growth in suspension culture, Graham et al., J. Gen. Virol., 36:59, 1977; baby hamster kidney cells (BHK, ATCC CCL 10); chinese hamster ovary-cells-DHFR (CHO, Urlaub and Chasin. Proc. Natl. Acad. Sci. 77; 4216, 1980); mouse sertoli cells (Mather, Biol. Reprod. 23:243-251, 1980); mouse fibroblast cells (NIH-3T3), monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI38, ATCC CCL 75); human liver cells (Hep G2, HB 8065); and mouse mammary tumor cells (MMT 060562, ATCC CCL51). Exemplary cancer cell types used for demonstrating the utility of recombinant cells (producing HPBF-peptide complexes) as a cancer vaccine are provided as follows: mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC#2 and SCLC#7.

A number of viral-based expression systems may also be utilized with mammalian cells to produce HSP peptide-binding fragments. Vectors using DNA virus backbones have been derived from simian virus 40 (SV40) (Hamer et al., 1979, Cell 17:725), adenovirus (Van Doren et al., 1984, Mol Cell Biol 4:1653), adeno-associated virus (McLaughlin et al., 1988, J Virol 62:1963), and bovine papillomas virus (Zinn et al., 1982, Proc Natl Acad Sci 79:4897). In cases where an adenovirus is used as an expression vector, the donor DNA sequence may be ligated to an adenovirus transcription/translation control region, e.g., the late promoter and tripartite leader sequence. This chimeric gene may then be inserted in the adenovirus genome by in vitro or in vivo recombination. Insertion in a non-essential region of the viral genome (e.g., region El or E3) will result in a recombinant virus that is viable and capable of expressing heterologous products in infected hosts (see e.g., Logan and Shenk, 1984, Proc. Natl. Acad. Sci. (USA) 81:3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copies/cell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene construct is transfected into cultured mammalian cells, for example, by the techniques of calcium phosphate coprecipitation or electroporation. For those host cells that do not manifest a transformed phenotype, selection of transformants is achieved by use of a dominant selectable marker, such as histidinol and G418 resistance. For example, BPV vectors such as pBCMGSNeo and pBCMGHis may be used to express HPBF sequences (Karasuyama et al., Eur. J. Immunol. 18:97-104; Ohe et al., Human Gene Therapy, 6:325-33) which may then be transfected into a diverse range of cell types for expression of the HPBF.

Alternatively, the vaccinia 7.5K promoter may be used (see, e.g., Mackett et al., 1982, Proc. Natl. Acad. Sci. (USA) 79:7415-7419; Mackett et al., 1984, J. Virol. 49:857-864; Panicali et al., 1982, Proc. Natl. Acad. Sci. 79:4927-4931.) In cases where a human host cell is used, vectors based on the Epstein-Barr virus (EBV) origin (OriP) and EBV nuclear antigen 1 (EBNA-1; a trans-acting replication factor) may be used. Such vectors can be used with a broad range of human host cells, e.g., EBO-pCD (Spickofsky et al., 1990, DNA Prot Eng Tech 2:14-18), pDR2 and λDR2 (available from Clontech Laboratories).

HPBFs may also be made with a retrovirus-based expression system. In contrast to transfection, retroviruses can efficiently infect and transfer genes to a wide range of cell types including, for example, primary hematopoietic cells. In retroviruses such as Moloney murine leukemia virus, most of the viral gene sequences can be removed and replaced with nucleic acid sequences encoding the HSP peptide-binding domain, while the missing viral functions can be supplied in trans. The host range for infection by a retroviral vector can also be manipulated by the choice of envelope used for vector packaging.

For example, a retroviral vector can comprise a 5′ long terminal repeat (LTR), a 3′ LTR, a packaging signal, a bacterial origin of replication, and a selectable marker. The HPBF DNA is inserted into a position between the 5′ LTR and 3′ LTR, such that transcription from the 5′ LTR promoter transcribes the cloned DNA. The 5′ LTR comprises a promoter, including but not limited to an LTR promoter, an R region, a U5 region and a primer binding site, in that order. Nucleotide sequences of these LTR elements are well known in the art. A heterologous promoter as well as multiple drug selection markers may also be included in the expression vector to facilitate selection of infected cells (see McLauchlin et al., 1990, Prog. Nucleic Acid Res. and Molec. Biol. 38:91-135; Morgenstern et al., 1990, Nucleic Acid Res. 18:3587-3596; Choulika et al., 1996, J. Virol 70:1792-1798; Boesen et al., 1994, Biotherapy 6:291-302; Salmons and Gunzberg, 1993, Human Gene Therapy 4:129-141; and Grossman and Wilson, 1993, Curr. Opin. in Genetics and Devel. 3:110-114).

Other usefil eukaryotic host-vector system may include yeast and insect systems. In yeast, a number of vectors containing constitutive or inducible promoters may be used with Saccharomyces cerevisiae (baker's yeast), Schizosaccharomyces pombe (fission yeast), Pichia pastoris, and Hansenula polymorpha (methylotropic yeasts). For a review see, “Current Protocols in Molecular Biology”, Vol. 2, 1988, Ausubel et al. (eds.), Greene Publish. Assoc. & Wiley Interscience, Ch. 13; Grant et al., 1987, Expression and Secretion Vectors for Yeast, 1987, in “Methods in Enzymology”, Wu and Grossman (eds.), Acad. Press, N.Y., Vol. 153, pp. 516-544; Glover, 1986, DNA Cloning, Vol. 11, IRL Press, Wash., D.C., Ch. 3; and Bitter, 1987, Heterologous Gene Expression in Yeast, in “Methods in Enzymology”, Berger and Kimmel (eds.), Acad. Press, N.Y., Vol. 152, pp. 673-684; and “The Molecular Biology of the Yeast Saccharomyces”, 1982, Strathern et al. (eds.), Cold Spring Harbor Press, Vols. I and II.

In an insect system a baculovirus, Autographa californica nuclear polyhidrosis virus (AcNPV), can be used as a vector to express an HPBF in Spodoptera frugiperda cells. The HPBF DNA may be cloned into non-essential regions (for example the polyhedrin gene) of the virus and placed under control of an AcNPV promoter (for example the polyhedrin promoter). These recombinant viruses are then used to infect host cells in which the inserted DNA is expressed (see, e.g., Smith et al., 1983, J. Virol. 46:584; Smith, U.S. Pat. No. 4,215,051).

Any of the cloning and expression vectors described herein may be synthesized and assembled from known DNA sequences by techniques well known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

5.2.2 Synthetic Production of HSP Peptide-Binding Fragments

An alternative to producing HPBF by recombinant techniques is peptide synthesis. For example, a peptide corresponding to a portion of an HSP comprising the substrate-binding domain, or which binds peptides in vitro, can be synthesized by use of a peptide synthesizer. Conventional peptide synthesis may be used or other synthetic protocols well known in the art.

In addition, analogs and derivatives of HPBF protein can be chemically synthesized. Furthermore, if desired, nonclassical amino acids or chemical amino acid analogs can be introduced as a substitution or addition into the HSP sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

Peptides having the amino acid sequence of a peptide-binding HPBF, or a mutant or derivative thereof, may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deparotected amino acid to an a-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting HSP peptide-binding fragment is accomplished using conventional procedures, such as preparative HPLC using gel permeation, partition and/or ion exchange chromatography. The choice of appropriate matrices and buffers are well known in the art and so are not described in detail herein.

5.2.3 Production of HSP Peptide-binding Fragments by Proteolytic Cleavage

In an alternative embodiment, peptide-binding HSP fragments may be obtained by chemical or enzymatic cleavage of native or recombinant HSPs. Specific chemical cleavage can be performed by cyanogen bromide, NaBH₄, acetylation, formylation, oxidation, reduction, metabolic synthesis in the presence of tunicamycin, etc. Endoproteases that cleave at specific sites can also be used. Such proteases are known in the art, including, but not limited to, trypsin, α-chymotrypsin, V8 protease, papain, and proteinase K (see Ausubel et al., (eds.), in “Current Protocols in Molecular Biology”, Greene Publishing Associates and Wiley Interscience, New York, 17.4.6-17.4.8). The amino acid sequence of an HSP of interest can be examined for the recognition sites of these proteases. An enzyme is chosen which can release a peptide-binding domain or peptide-binding fragment. The HSP molecule is then incubated with the protease, under conditions that allow digestion by the protease and release of the specifically designated peptide-binding fragments. Alternatively, such protease digestions can be carried out blindly, i.e., not knowing which digestion product will contain the peptide-binding domain, using specific or general specificity proteases, such as proteinase K or pronase. The digestion products may be purified as described above, and subsequently tested for the ability to bind peptide or for immunogenicity. Methods for determining the immunogenicity of HSP complexes by cytotoxicity tests are described in Section 5.4.1.

5.3 HSP Peptide-binding Fragments - Antigenic Molecule Complexes

5.3.1 Preparation of Intracellular Complexes of HSP Peptide-Binding Fragments with Antigenic Molecules

Described herein are methods for purifying HSP peptide-binding fragments or HPBF-peptide complexes of the invention from recombinant cells, and, with minor modifications known in the art, the HSP peptide-binding fragment or fragment-peptide complexes from the cell culture. Recombinant cells include, for example, cells expressing antigenic molecules and recombinantly expressing heat shock protein or an HPBF. Such cells may be derived from a variety of sources, including, but not limited to, cells infected with an infectious agent and cancer cells.

The purification of HSP-peptide complexes from cell lysates has been described previously, see, for example, Udono et al., 1993, J. Exp. Med. 178:1391-1396. The purification of Hsp90-peptide complexes and gp96-peptide complexes from cell lysates have been described, for example, in WO 95/24923, dated Sep. 21, 1995, and WO 97/10000, dated Mar. 20, 1997, which are incorporated herein by reference in their entirety.

The invention provides methods for purification of recombinant HPBF-peptide complexes by affinity purification, based on the properties of the affinity label present on the HPBF. One approach is based on specific molecular interactions between a tag and its binding partner. The other approach relies on the immunospecific binding of an antibody to an epitope present on the tag. The principle of affinity chromatography well known in the art is generally applicable to both of these approaches.

To produce HPBF-antigenic peptide complexes, a nucleotide sequence encoding an HPBF can be introduced into a cell. When an antigenic molecule is present in the cell, the HPBF can associate intracellularly with the antigenic molecule, forming a non-covalent complex of HPBF and the antigenic molecule. Cells into which an HPBF-encoding nucleotide sequence can be introduced, include, but are not limited to, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. The choice of cell type depends on the type of tumor or infectious disease being treated or prevented, and can be determined by one of skill in the art.

In a specific embodiment, an expression construct comprising a nucleic acid sequence encoding the HSP peptide-binding fragment is introduced into an antigenic cell. As used herein, antigenic cells may include cells that are infected with an infectious agent or pathogen, cells infected with non-infectious or non-pathogenic forms of an infectious agent or pathogen (e.g., by use of a helper infectious agent), cells infected by or engineered to express an attenuated form of an infectious agent or a non-pathogenic or replication-deficient variant of a pathogen, pre-neoplastic cells that are infected with a cancer-causing infectious agent, such as a virus, but which are not yet neoplastic; or antigenic cells that have been exposed to a mutagen or cancer-causing agent, such as, for example DNA-damaging agents, radiation, etc. Other cells that can be used are pre-neoplastic cells which are in transition from a normal to a neoplastic form as characterized by morphology, physiological or biochemical functions. Preferably, the cancer cells and pre-neoplastic cells used in the methods of the invention are of mammalian origin. Mammals contemplated by this aspect of the invention include humans, companion animals (e.g., dogs and cats), livestock animals (e.g., sheep, cattle, goats, pigs and horses), laboratory animals (e.g., mice, rats and rabbits), and captive or free wild animals.

In various embodiments, any cancer cell, preferably a human cancer cell, can be used in the present methods for producing HPBF-peptide complexes. The cancer cells provide the antigenic peptides which become associated noncovalently with the expressed HPBF. HPBF-antigenic peptide complexes are then purified from the cells and used to treat such cancers. Cancers which can be treated or prevented with immunogenic compositions prepared by methods of the invention include, but are not limited to, tumors such as sarcomas and carcinomas. Examples of cancers that are amenable to the methods of the invention are listed in Section 5.4.2. Accordingly, any tissues or cells isolated from a pre-neoplastic lesion, a cancer, including cancer that has metastasized to multiple remote sites, can be used in the present method. For example, cells found in abnormally growing tissue, circulating leukemic cells, metastatic lesions as well as solid tumor tissue can be used.

In another embodiment, cell lines derived from a pre-neoplastic lesion, cancer tissues or cancer cells can also be used, provided that the cells of the cell line have at least one or more antigenic determinants in common with antigens on the target cancer cells. Cancer tissues, cancer cells, cells infected with a cancer-causing agent, other pre-neoplastic cells, and cell lines of human origin are preferred.

Cancer and pre-neoplastic cells can be identified by any method known in the art. For example, cancer cells can be identified by morphology, enzyme assays, proliferation assays, cytogenetic characterization, DNA mapping, DNA sequencing, the presence of cancer-causing virus, or a history of exposure to mutagen or cancer-causing agent, imaging, etc. Cancer cells may also be obtained by surgery, endoscopy, or other biopsy techniques. If some distinctive characteristics of the cancer cells are known, they can also be obtained or purified by any biochemical or immunological methods known in the art, such as but not limited to affinity chromatography, and fluorescence activated cell sorting (e.g., with fluorescently tagged antibody against an antigen expressed by the cancer cells).

Cancer tissues, cancer cells or cell lines may be obtained from a single individual or pooled from several individuals. It is not essential that clonal, homogeneous, or purified population of cancer cells be used. It is also not necessary to use cells of the ultimate target in vivo (e.g., cells from the tumor of the intended recipient), so long as at least one or more antigenic determinants on the target cancer cells is present on the cells used for expression of the HPBF. In addition, cells derived from distant metastases may be used to prepare an immunogenic composition against the primary cancer. A mixture of cells can be used provided that a substantial number of cells in the mixture are cancer cells and share at least one antigenic determinant with the target cancer cell. In a specific embodiment, the cancer cells to be used in expressing an HPBF are purified.

5.3.1.1 Preparation of Hsp70-Peptide Complexes

The purification of Hsp70-peptide complexes has been described previously, see, for example, Udono et al., 1993, J. Exp. Med. 178:1391-1396. The following procedure may be used, presented by way of example but not limitation, to purify Hsp70 complexes. Initially, tumor cells are suspended in 3 volumes of IX Lysis buffer consisting of 5 mM sodium phosphate buffer, pH 7, 150 mM NaCl, 2 mM CaCl₂, 2 mM MgCl₂ and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate pH 7.5, 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose equilibrated with phosphate buffered saline (PBS) containing 2 mM Ca²⁺ and 2 mM Mg²⁺. When the cells are lysed by mechanical shearing the supernatant is diluted with an equal volume of 2X lysis buffer prior to mixing with Con A Sepharose. The supernatant is then allowed to bind to the Con A Sepharose for 2-3 hours at 4° C. The material that fails to bind is harvested and dialyzed for 36 hours (three times, 100 volumes each time) against 10 mM Tris-Acetate pH 7.5, 0.1 mM EDTA, 10 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC column equilibrated in 20 mM Tris-acetate pH 7.5, 2 mM NaCl, 0.1 mM EDTA and 15 mM 2-mercaptuethanol). The column is then developed with a 20 mM to 500 mM NaCl gradient and then eluted fractions fractionated by sodium dodecyl sulfate-polyacrylamide gel electrophoresis (SDS-PAGE) and characterized by immunoblotting using an appropriate anti-Hsp70 antibody (such as from clone N27F3-4, from StressGen).

Fractions strongly immunoreactive with the anti-Hsp70 antibody are pooled and the Hsp70 -peptide complexes precipitated with ammonium sulfate; specifically with a 50%-70% ammonium sulfate cut. The resulting precipitate is then harvested by centrifugation at 17,000 rpm (SS34 Sorvall rotor) and washed with 70% ammonium sulfate. The washed precipitate is then solubilized and any residual ammonium sulfate removed by gel filtration on a Sephadex^{R} G25 column (Pharmacia). If necessary the Hsp70 preparation thus obtained can be repurified through the Mono Q FPLC Column as described above.

The Hsp70-peptide complex can be purified to apparent homogeneity using this method. Typically 1 mg of Hsp70 -peptide complex can be purified from 1 g of cells/tissue.

An improved method for purification of Hsp70-peptide complexes comprises contacting cellular proteins with ADP or a nonhydrolyzable analog of ATP affixed to a solid substrate, such that Hsp70 in the lysate can bind to the ADP or nonhydrolyzable ATP analog, and eluting the bound Hsp70. A preferred method uses column chromatography with ADP affixed to a solid substratum (e.g., ADP-agarose). The resulting Hsp70 preparations are higher in purity and devoid of non-specifically bound peptides. The Hsp70 yields are also increased significantly by about more than 10 fold. Alternatively, chromatography with nonhydrolyzable analogs of ATP, instead of ADP, can be used for purification of Hsp70-peptide complexes. By way of example but not limitation, purification of Hsp70-peptide complexes by ADP-agarose chromatography can be carried out as follows:

Meth A sarcoma cells (500 million cells) are homogenized in hypotonic buffer and the lysate is centrifuged at 100,000 g for 90 minutes at 4° C. The supernatant is applied to an ADP-agarose column. The column is washed in buffer and is eluted with 5 column volumes of 3 mM ADP. The Hsp70 peptide complexes elute in fractions 2 through 10 of the total 15 fractions which elute. The eluted fractions are analyzed by SDS-PAGE. The Hsp70-peptide complexes can be purified to apparent homogeneity using this procedure.

5.3.1.2 Preparation of Hsp90-Peptide Complexes

The purification of Hsp90-peptide complexes from cell lysates has been described, for example, in WO 95/24923, dated Sep. 21, 1995, and WO 97/10000, dated Mar. 20, 1997. A procedure that can be used, presented by way of example and not limitation, is as follows:

Initially, tumor cells are suspended in 3 volumes of IX Lysis buffer consisting of 5 mM sodium phosphate buffer (pH7), 150 mM NaCl, 2 mM CaCl₂, 2 mM MgCl₂ and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate pH 7.5, 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifiged at 100,000 g for 90 minutes, the supernatant harvested and then mixed with Con A Sepharose equilibrated with PBS containing 2 mM Ca²⁺ and 2 mM Mg²⁺. When the cells are lysed by mechanical shearing the supernatant is diluted with an equal volume of 2X Lysis buffer prior to mixing with Con A Sepharose. The supernatant is then allowed to bind to the Con A Sepharose for 2-3 hours at 4° C. The material that fails to bind is harvested and dialyzed for 36 hours (three times, 100 volumes each time) against 20 mM Sodium phosphate pH 7.4, 1 mM EDTA, 250 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC column equilibrated with dialysis buffer. The proteins are then eluted with a salt gradient of 200 mM to 600 mM NaCl.

The eluted fractions are fractionated by SDS-PAGE and fractions containing the Hsp90-peptide complexes identified by immunoblotting using an anti-Hsp90 antibody such as 3G3 (Affinity Bioreagents). Hsp90-peptide complexes can be purified to apparent homogeneity using this procedure. Typically, 150-200 μg of Hsp90-peptide complex can be purified from 1 g of cells/tissue.

5.3.1.3 Preparation of gp96-Peptide Complexes

The purification of gp96-peptide complexes from cell lysates has been described, for example, in WO 95/24923, dated Sep. 21, 1995, and WO 97/10000, dated Mar. 20, 1997. A procedure that can be used, presented by way of example and not limitation, is as follows:

A cell pellet is resuspended in 3 volumes of buffer consisting of 30 mM sodium bicarbonate buffer (pH 7.5) and 1 mM PMSF and the cells allowed to swell on ice 20 minutes. The cell pellet is then homogenized in a Dounce homogenizer (the appropriate clearance of the homogenizer will vary according to each cell type) on ice until >95% cells are lysed.

The lysate is centrifuged at 1,000 g for 10 minutes to remove unbroken cells, nuclei and other debris. The supernatant from this centrifugation step is then recentrifuged at 100,000 g for 90 minutes. The gp96-peptide complex can be purified either from the 100,000 pellet or from the supernatant.

When purified from the supernatant, the supernatant is diluted with equal volume of 2X lysis buffer and the supernatant mixed for 2-3 hours at 4° C. with ConA Sepharose equilibrated with PBS containing 2 mM Ca²+and 2 mM Mg²⁺. Then, the slurry is packed into a column and washed with 1X lysis buffer until the OD₂₈₀ drops to baseline. Then, the column is washed with ⅓ column bed volume of 10% α-methyl mannoside (α-MM) dissolved in PBS containing 2 mM Ca²⁺ and 2 mM Mg²⁺, the column sealed with a piece of parafilm, and incubated at 37° C. for 15 minutes. Then the column is cooled to room temperature and the parafilm removed from the bottom of the column. Five column volumes of the α-MM buffer are applied to the column and the eluate analyzed by SDS-PAGE. Typically the resulting material is about 60-95% pure, however this depends upon the cell type and the tissue-to-lysis buffer ratio used. Then the sample is applied to a Mono Q FPLC column (Pharmacia) equilibrated with a buffer containing 20 mM sodium phosphate, pH 7.4. The proteins are then eluted from the column with a 0-1M NaCl gradient and the gp96 fraction elutes between 400 mM and 550 mM NaCl.

The procedure, however, may be modified by two additional steps, used either alone or in combination, to consistently produce apparently homogeneous gp96-peptide complexes. One optional step involves an ammonium sulfate precipitation prior to the Con A purification step and the other optional step involves DEAE-Sepharose purification after the Con A purification step but before the Mono Q FPLC step.

In the first optional step, described by way of example as follows, the supernatant resulting from the 100,000 g centrifugation step is brought to a final concentration of 50% ammonium sulfate by the addition of ammonium sulfate. The ammonium sulfate is added slowly while gently stirring the solution in a beaker placed in a tray of ice water. The solution is stirred from about ½ to 12 hours at 4° C. and the resulting solution centrifuged at 6,000 rpm (Sorvall SS34 rotor). The supernatant resulting from this step is removed, brought to 70% ammonium sulfate saturation by the addition of ammonium sulfate solution, and centrifuged at 6,000 rpm (Sorvall SS34 rotor). The resulting pellet from this step is harvested and suspended in PBS containing 70% ammonium sulfate in order to rinse the pellet. This mixture is centrifuged at 6,000 rpm (Sorvall SS34 rotor) and the pellet dissolved in PBS containing 2 mM Ca²⁺ and Mg²⁺. Undissolved material is removed by a brief centrifugation at 15,000 rpm (Sorvall SS34 rotor). Then, the solution is mixed with Con A Sepharose and the procedure followed as before.

In the second optional step, described by way of example as follows, the gp96 containing fractions eluted from the Con A column are pooled and the buffer exchanged for 5 mM sodium phosphate buffer, pH 7, 300 mM NaCl by dialysis, or preferably by buffer exchange on a Sephadex G25 column. After buffer exchange, the solution is mixed with DEAE-Sepharose previously equilibrated with 5 mM sodium phosphate buffer, pH 7, 300 mM NaCl. The protein solution and the beads are mixed gently for 1 hour and poured into a column. Then, the column is washed with 5 mM sodium phosphate buffer, pH 7, 300 mM NaCl, until the absorbance at 280 nm drops to baseline. Then, the bound protein is eluted from the column with five volumes of 5 mM sodium phosphate buffer, pH 7, 700 mM NaCl. Protein containing fractions are pooled and diluted with 5 mM sodium phosphate buffer, pH 7 in order to lower the salt concentration to 175 mM. The resulting material then is applied to the Mono Q FPLC column (Pharmacia) equilibrated with 5 mM sodium phosphate buffer, pH 7 and the protein that binds to the Mono Q FPLC column (Pharmacia) is eluted as described before.

It is appreciated, however, that one skilled in the art may assess, by routine experimentation, the benefit of incorporating the second optional step into the purification protocol. In addition, it is appreciated also that the benefit of adding each of the optional steps will depend upon the source of the starting material.

When the gp96 fraction is isolated from the 100,000 g pellet, the pellet is suspended in 5 volumes of PBS containing either 1% sodium deoxycholate or 1% octyl glucopyranoside (but without the Mg²⁺ and Ca²⁺) and incubated on ice for 1 hour. The suspension is centrifuged at 20,000 g for 30 minutes and the resulting supernatant dialyzed against several changes of PBS (also without the Mg²⁺ and Ca²⁺) to remove the detergent. The dialysate is centrifuged at 100,000 g for 90 minutes, the supernatant harvested, and calcium and magnesium are added to the supernatant to give final concentrations of 2 mM, respectively. Then the sample is purified by either the unmodified or the modified method for isolating gp96-peptide complex from the 100,000 g supernatant, see above.

The gp96-peptide complexes can be purified to apparent homogeneity using this procedure. About 10-20 gg of gp96 can be isolated from 1 g cells/tissue.

5.3.2 In Vitro Complexing

In another embodiment, complexes of HPBFs to antigenic molecules are produced in vitro. Immunogenic HPBF-peptide complexes can be generated in vitro by noncovalent coupling of an HPBF with an antigenic peptide. Antigenic molecules may be isolated from various sources, chemically synthesized, or produced recombinantly. Procedures for forming such HSP-peptide complexes and methods for isolating antigenic peptides are described in detail herein. Such methods can be readily adapted for medium or large scale production of the immunotherapeutic or prophylactic vaccines of the invention.

A preferred, exemplary protocol for noncovalently complexing an HPBF and an antigenic molecule in vitro is provided herein.

It may be advantageous to use HPBFs that are reversibly bound to a solid phase to facilitate buffer exchange, washings and isolation of the complexes before or after the complexing reaction. Prior to complexing, the HPBFs may be pretreated with ATP or low pH to remove any peptides that may be associated with the HPBF of interest. When the ATP procedure is used, excess ATP is removed from the preparation by the addition of apyranase as described by Levy et al., 1991, Cell 67:265-274. When the low pH procedure is used, the buffer is readjusted to neutral pH by the addition of pH modifying reagents.

The antigenic molecules (1 μg) and the pretreated HPBF (9 μg) are admixed to give an approximately 5:1 antigenic molecule: HPBF molar ratio. Then, the mixture is incubated for 15 minutes to 3 hours at 25° C. to 45° C. in a suitable binding buffer such as one containing 20 mM sodium phosphate, pH 7.2, 350 mM NaCl, 3 mM MgCl₂ and 1 mM phenyl methyl sulfonyl fluoride (PMSF). The preparations are centrifuged through a Centricon 10 assembly (Millipore) to remove any unbound peptide. If the HPBF is bound to a solid phase, the HPBF-peptide complexes formed can be washed free of unbounded peptide prior to eluting the HPBF-peptide complex off the solid phase. The association of the peptides with the HPBF can be assayed by SDS-PAGE. This is the preferred method for in vitro complexing of peptides isolated from MHC-peptide complexes of peptides disassociated from endogenous HSP-peptide complexes.

In an alternative embodiment, preferred for producing complexes of Hsp70 fragments to exogenous antigenic molecules such as proteins, 5-10 micrograms of purified HPBF is incubated with equimolar quantities of the antigenic molecule in 20 mM sodium phosphate buffer pH 7.5, 0.5 M NaCl, 3 mM MgCl₂ and 1 mM ADP in a volume of 100 microliter at 37° C. for 1 hr. This incubation mixture is further diluted to 1 ml in phosphate-buffered saline.

In another alternative embodiment of the invention, preferred for producing complexes of gp96 fragment to peptides, 5-10 micrograms of gp96 fragment immobilized by its affinity tag to a solid phase is incubated with equimolar or excess quantities of the antigenic peptide in a suitable buffer, such as one containing 20 mM sodium phosphate buffer pH 7.5, 0.5 M NaCl, 3 nM MgCl2 at about 50° C. for about 10 minutes. For example, modified gp96 containing the Ig tag can be immobilized to protein A-Sepharose for this procedure. This incubation mixture is then further incubated for about 30 minutes at room temperature. The solid phase with the bound HPBF-peptide complexes is washed several times to remove any unbound peptide. The HPBF-peptide complexes is then eluted from the solid phase by the appropriate technique.

Following complexing, the immunogenic HSP-antigenic molecule complexes can optionally be assayed in vitro using, for example, the mixed lymphocyte target cell assay (MLTC) described below. Once immunogenic complexes have been isolated they can be optionally characterized further in animal models using the preferred administration protocols and excipients discussed below.

5.3.3 Sources of Antigenic Molecules

Antigens, or antigenic portions thereof, specific to one or more types of cancer or infected cells, can be chosen from among those known in the art. Alternatively, such antigens can be selected for their antigenicity or their immunogenicity, as determined by immunoassays or by their ability to generate an immune response.

5.3.3.1 Exogenous Antigenic Molecules

Preferably, where it is desired to treat or prevent cancer, known tumor-specific antigens or fragments or derivatives thereof are used. For example, such tumor specific or tumor-associated antigens include but are not limited to KS ¼ pan-carcinoma antigen (Perez and Walker, 1990, J. Immunol. 142:3662-3667; Bumal, 1988, Hybridoma 7(4):407-415); ovarian carcinoma antigen (CA125) (Yu, et al, 1991, Cancer Res. 51(2):468-475); prostatic acid phosphate (Tailer, et al., 1990, Nucl. Acids Res. 18(16):4928); prostate specific antigen (Henttu and Vihko, 1989, Biochem. Biophys. Res. Comm. 160(2):903-910; Israeli, et al., 1993, Cancer Res. 53:227-230); melanoma-associated antigen p97 (Estin, et al., 1989, J. Natl. Cancer Inst. 81(6):445-446); melanoma antigen gp75 (Vijayasardahl, et al., 1990, J. Exp. Med. 171(4):1375-1380); high molecular weight melanoma antigen (Natali, et al., 1987, Cancer 59:55-63) and prostate specific membrane antigen.

In a specific embodiment, an antigen or fragment or derivative thereof specific to a certain tumor is selected for complexing to HPBF and subsequent administration to a patient having that tumor.

In a preferred embodiment, where it is desired to treat or prevent viral diseases, molecules comprising epitopes of known viruses are used. For example, such antigenic epitopes may be prepared from viruses including, but not limited to, hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

In another preferred embodiment, where it is desired to treat or prevent bacterial infections, molecules comprising epitopes of known bacteria are used. For example, such antigenic epitopes may be prepared from bacteria including, but not limited to, mycobacteria rickettsia, mycoplasma, neisseria and legionella.

In another preferred embodiment, where it is desired to treat or prevent protozoal infections, molecules comprising epitopes of known protozoa are used. For example, such antigenic epitopes may be prepared from protozoa including, but not limited to, leishmania, kokzidioa, and trypanosoma.

In yet another preferred embodiment, where it is desired to treat or prevent parasitic infections, molecules comprising epitopes of known parasites are used. For example, such antigenic epitopes may be from parasites including, but not limited to, chlamydia and rickettsia.

To determine immunogenicity or antigenicity of a putative antigen by detecting binding to antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, etc. In one aspect, antibody binding is detected by detecting a label on the primary antibody. In another aspect, the primary antibody is detected by detecting binding of a secondary antibody or reagent to the primary antibody. In a further aspect, the secondary antibody is labeled. Many means are known in the art for detecting binding in an immunoassay and are envisioned for use. In one embodiment for detecting immunogenicity, T cell-mediated responses can be assayed by standard methods, e.g., in vitro cytoxicity assays or in vivo delayed-type hypersensitivity assays.

Potentially useful antigens, or derivatives thereof, can be identified by various criteria, such as the antigen's involvement in neutralization of a pathogen's infectivity (wherein it is desired to treat or prevent infection by such a pathogen) (Norrby, 1985, Summary, in Vaccines 85, Lemer, et al. (eds.), Cold Spring Harbor Laboratory, Cold Spring Harbor, N.Y., pp. 388-389), type or group specificity, recognition by patients' antisera or immune cells, and/or the demonstration of protective effects of antisera or immune cells specific for the antigen. In addition, where it is desired to treat or prevent a disease caused by pathogen, the antigen's encoded epitope should preferably display a small or no degree of antigenic variation in time or amongst different isolates of the same pathogen.

5.3.3.2 Peptides From Hsp-Peptide Complexes

Antigenic peptides for complexing in vitro to HPBFs of the invention can also be obtained from endogenous complexes of peptides and HSPs. Two methods may be used to elute the peptide from an HSP-peptide complex. One approach involves incubating the HSP-peptide complex in the presence of ATP. The other approach involves incubating the complexes in a low pH buffer.

Briefly, the complex of interest is centrifuged through a Centricon 10 assembly (Millipore) to remove any low molecular weight material loosely associated with the complex. The large molecular weight fraction may be removed and analyzed by SDS-PAGE while the low molecular weight may be analyzed by HPLC as described below. In the ATP incubation protocol, the HSP-peptide complex in the large molecular weight fraction is incubated with 10 mM ATP for 30 minutes at room temperature. In the low pH protocol, acetic acid or trifluoroacetic acid (TFA) is added to the HSP-peptide complex to give a final concentration of 10% (vol/vol) and the mixture incubated at room temperature or in a boiling water bath or any temperature in between, for 10 minutes (See, Van Bleek, et al., 1990, Nature 348:213-216; and Li, et al., 1993, EMBO Journal 12:3143-3151).

The resulting samples are centrifuged through a Centricon 10 assembly as mentioned previously. The high and low molecular weight fractions are recovered. The remaining large molecular weight HSP-peptide complexes can be reincubated with ATP or low pH to remove any remaining peptides.

The resulting lower molecular weight fractions are pooled, concentrated by evaporation and dissolved in 0.1 % TFA. The dissolved material is then fractionated by reverse phase high pressure liquid chromatography (HPLC) using for example a VYDAC C 18 reverse phase column equilibrated with 0.1 % TFA. The bound material is then eluted at a flow rate of about 0.8 ml/min by developing the column with a linear gradient of 0 to 80% acetonitrile in 0.1% TFA. The elution of the peptides can be monitored by OD₂₁₀ and the fractions containing the peptides collected.

5.3.3.3 Peptides from MHC-Peptide Complexes

Peptides bound to MUC molecules in vivo can also be used in vitro to form complexes with HPBFs of the invention. The isolation of potentially immunogenic peptides from MHC molecules is well known in the art and so is not described in detail herein (see, Falk, et al., 1990, Nature 348:248-251; Rotzsche, at al., 1990, Nature 348:252-254; Elliott, et al., 1990, Nature 348:191-197; Falk, et al., 1991, Nature 351:290-296; Demotz, et al., 1989, Nature 343:682-684; Rotzsche, et al., 1990, Science 249:283-287), the disclosures of which are incorporated herein by reference.

Briefly, MHC-peptide complexes may be isolated by a conventional immunoaffinity procedure. The peptides then may be eluted from the MHC-peptide complex by incubating the complexes in the presence of about 0.1 % TFA in acetonitrile. The eluted peptides may be fractionated and purified by reverse phase HPLC, as before.

5.3.3.4 Synthetic Peptides

The amino acid sequences of the peptides eluted from MHC molecules or HSPs may be determined either by manual or automated amino acid sequencing techniques well known in the art. Once the amino acid sequence of a potentially protective peptide has been determined, the peptide may be synthesized in using conventional peptide synthesis or other protocols well known in the art.

Peptides having the same amino acid sequence as those isolated above may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting peptides is accomplished using conventional procedures, such as preparative HPLC using gel permeation, partition and/or ion exchange chromatography. The choice of appropriate matrices and buffers are well known in the art and so are not described in detail herein.

5.3.3.5 Recombinantly Produced Antigens

In a particular embodiment of the invention, a nucleotide sequence encoding a protein antigen or portions thereof can be introduced into a host cell for production of the antigen. The nucleotide sequence encoding any antigenic protein can be obtained and cloned into an expression vector for expression essentially by the same methods described for the cloning and expression of a nucleotide sequence encoding an HSP peptide-binding fragment. The techniques are described in Sections 5.2.1.1 and 5.2.1.2 and are well known in the art. The recombinant antigenic protein or portions thereof can be purified by any methods appropriate for the protein, and then used to form complexes with HPBFs in vitro as described in Section 5.3.2. Such an HPBF-antigen complex can be used as a vaccine to stimulate an immune response against the antigenic protein in a subject for the purpose of treatment or prevention of infectious diseases or cancer.

5.4 Therapeutic Applications of Noncovalent Complexes of HSP Peptide-Binding Fragments and Antigenic Molecules

The present invention encompasses the use of HPBFs in methods for treatment of and prevention of infectious diseases and cancer. In various embodiments described in detail herein, an effective amount of a HSP peptide-binding fragment in a noncovalent complex with an antigenic molecule is administered to a patient for therapeutic purposes.

5.4.1 Prevention and Treatment of Infectious Diseases

For treatment and prevention of infectious disease, HPBF complexes are prepared from a cell that displays the antigenicity of an antigen of an infectious agent or pathogenic agent, and used as vaccines against the infectious disease. As will be appreciated by those skilled in the art, the protocols described herein may be used to isolate HPBF-peptide complexes from any cell that displays the antigenicity of an antigen of the infectious agent. For example, cells may be infected by the infectious agent itself, or alternatively, cells may be infected by or engineered to express an attenuated form of the infectious agent or a non-pathogenic or replication-deficient variant of the pathogen. In one embodiment, HPBF complexes can be prepared from cells infected with non-infectious or non-pathogenic forms of the infectious agent (e.g., by use of a helper infectious agent). In another embodiment, the HPBF complexes of the invention may be prepared from cells infected with an intracellular pathogen. In another embodiment, HPBF-complexes can be prepared from cells that have been transformed by an intracellular pathogen. For example, immunogenic HSP peptide-binding fragments complexes may be isolated from eukaryotic cells transformed with a transforming virus such as SV40.

A preferred method for treatment or prevention of an infectious disease comprises introducing into a cell that displays the antigenicity of an infectious agent an expressible HPBF gene sequence, preferably as an expression gene construct. The HPBF gene sequence is manipulated by methods described above in Section 5.2.1. 1, so that the HPBF gene sequence, in the form of an expression construct, located extrachromosomally or integrated in the chromosome, is suitable for expression of the HPBF in the recombinant cells. The recombinant cells containing the expression gene constructs are cultured under conditions such that HPBFs encoded by the expression gene construct are expressed. Complexes of HPBFs noncovalently associated with antigenic peptides of the infectious agent are purified from the cell culture or culture medium by the methods described in Section 5.2. Exemplary methods of therapeutic and prophylactic uses of HSP-peptide complexes against intracellular pathogens have also been described in PCT Publications WO 95/24923, dated Sep. 21, 1995, and WO 97/10001, dated Mar. 20, 1997.

In various embodiments, HPBF complexes are prepared from a cell genetically manipulated to express an HPBF, for example, tissues, isolated cells or immortalized eukaryotic cell lines infected with an intracellular pathogen. When immortalized animal cell lines are used as a source of the HPBF-peptide complex, it is important to use cell lines that can be infected with the pathogen of interest. In addition, it is preferable to use cells that are derived from the same species as the intended recipient of the vaccine. Techniques for introducing an expressible form of the HPBF gene sequences into these cell lines are described above in Section 5.3.1.1.

If a pathogen is expected to cause lysis of the host cells, it is preferred to introduce the expressible HPBF gene sequence into the host cell prior to infecting the cells with the pathogen. For example, in order to prepare an HPBF-peptide complex for administration to humans that may be effective against HIV-1, the virus may be propagated in human cells which include, but are not limited to, human CD4+T cells, HepG2 cells, and U937 promonocytic cells, which have already been transfected with an expressible HPBF sequence. Similarly, influenza viruses may be propagated in, for example, transfected human fibroblast cell lines and MDCK cells, and mycobacteria may be cultured in, for example, transfected human Schwaan cells. The cell supernatant containing HPBF-peptide complex may be collected just prior to lysis of the host cell.

In a preferred aspect of the invention, the purified HSP peptide-binding fragment complex vaccines may have particular utility in the treatment of human diseases caused by intracellular pathogens. It is appreciated, however, that the vaccines developed using the principles described herein will be useful in treating diseases of other mammals, for example, farm animals including: cattle; horses; sheep; goats; and pigs, and household pets including: cats; and dogs, that similarly are caused by intracellular pathogens.

In accordance with the methods described herein, vaccines may be prepared that stimulate an immune response, in particular a cytotoxic T cell responses, against cells infected with viruses including, but not limited to, hepatitis type A, hepatitis type B, hepatitis type C, influenza, varicella, adenovirus, HSV-I, HSV-II, rinderpest rhinovirus, echovirus, rotavirus, respiratory syncytial virus, papilloma virus, papova virus, cytomegalovirus, echinovirus, arbovirus, huntavirus, coxsachie virus, mumps virus, measles virus, rubella virus, polio virus, HIV-I, and HIV-II. Similarly, vaccines may also be prepared that stimulate cytotoxic T cell responses against cells infected with intracellular bacteria, including, but not limited to, Mycobacteria, Rickettsia, Mycoplasma, Neisseria and Legionella. In addition, vaccines may also be prepared that stimulate cytotoxic T cell responses against cells infected with intracellular protozoa, including, but not limited to, Leishmani, Kokzidioa, and Trypanosoma. Furthermore, vaccines may be prepared that stimulate cytotoxic T cell responses against cells infected with intracellular parasites including, but not limited to, Chlamydia and Rickettsia.

The effect of immunotherapy with modified HPBF-peptide complexes on progression of infectious diseases can be monitored by any methods known to one skilled in the art.

5.4.2 Prevention and Treatment of Cancer

There are many reasons why immunotherapy as provided by the noncovalent HPBF-peptide complexes or recombinant cells expressing HPBFs prepared by the present invention is desired for use in cancer patients. First, if cancer patients are immunosuppressed, and surgery with anesthesia, and subsequent chemotherapy, may worsen the immunosuppression, then with appropriate immunotherapy in the preoperative period, this immunosuppression may be prevented or reversed. This could lead to fewer infectious complications and to accelerated wound healing. Second, tumor bulk is minimal following surgery and immunotherapy is most likely to be effective in this situation. A third reason is the possibility that tumor cells are shed into the circulation at surgery and effective immunotherapy applied at this time can eliminate these cells.

In a specific embodiment, the preventive and therapeutic utility of the invention is directed at enhancing the immunocompetence of the cancer patient either before surgery, at or after surgery, and at inducing tumor-specific immunity to cancer cells, with the objective being inhibition of cancer, and with the ultimate clinical objective being total cancer regression and eradication.

According to the invention, preferred methods of treatment or prevention of cancer comprise isolating cancer cells from one or more individual, preferably the individual in need of treatment, and introducing into such cells an expressible HPBF gene sequence, preferably as an expression gene construct. The HPBF gene sequence is manipulated by methods described above in Section 5.2.1.1, such that the HPBF gene sequence, in the form of an expression construct, or intrachromosomally integrated, are suitable for expression of the HPBF in the recombinant cells. The recombinant cells containing the expression gene constructs are cultured under conditions such that HPBFs encoded by the expression gene construct are expressed by the recombinant host cells. Complexes of HPBFs noncovalently associated with peptides of the cancer cell are purified from the cell culture or culture medium by the methods described in Section 5.2. Depending on the route of administration, the HPBF-peptide complexes are formulated accordingly as described in Section 5.6, and administered to the individual autologously (e.g., to treat the primary cancer or metastases thereof), or to other individuals who are in need of treatment for cancer of a similar tissue type, or to individuals at enhanced risk of cancer due to familial history or environmental risk factors. Exemplary methods of therapeutic and prophylactic uses of HSP-peptide complexes have also been described in PCT Publications WO 96/10411, dated Apr. 11, 1996, and WO 97/10001, dated Mar. 20, 1997.

For example, treatment with HPBF-peptide complexes prepared as described above may be started any time after surgery. However, if the patient has received chemotherapy, HSP-antigen complexes are usually administered after an interval of four weeks or more so as to allow the immune system to recover. The therapeutic regimen may include weekly injections of the HPBF-antigen complex, dissolved in saline or other physiologically compatible solution. The route and site of injection is varied each time, for example, the first injection is given subcutaneously on the left arm, the second injection on the right arm, the third injection on the left abdominal region, the fourth injection on the right abdominal region, the fifth injection on the left thigh, the sixth injection on the right thigh, etc. The same site is repeated after a gap of one or more injections. In addition, injections are split and each half of the dose is administered at a different site on the same day. Overall, the first four to six injections are given at weekly intervals. Subsequently, two injections are given at two-week intervals, followed by a regimen of injections at monthly intervals.

Alternatively, recombinant tumor cells expressing HSP-peptide complexes can be used as a vaccine for injection into a patient to stimulate an immune response against the tumor cells or cells bearing tumor antigens. Autologous recombinant tumor cells stably expressing HPBF-peptide complexes are preferred. To determine the appropriate dose, the amount of HPBF-peptide complex produced by the recombinant cells is quantitated, and the number of recombinant cells used for vaccination is adjusted accordingly to assure a consistent level of expression in vivo. A preferred dose is the number of recombinant cells that can produce about 100 ng HPBF per 24 hours. For the safety of the patient, the recombinant tumor cells can be irradiated (12000 rad) immediately prior to injection into a patient. Irradiated cells do not proliferate, and can continue to express HPBF-peptide complexes for about 7-10 days which is sufficient to induce an immune response. Cancers that can be treated or prevented by using noncovalent HSP-peptide complexes prepared by the methods of the present invention include, but not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

In a specific embodiment, the cancer is metastatic. In another specific embodiment, the patient having a cancer is immunosuppressed by reason of having undergone anti-cancer therapy (e.g., chemotherapy radiation) prior to administration of the HSP-peptide molecule complexes of the invention. In another specific embodiment, the cancer is a tumor.

The effect of immunotherapy with HPBF-peptide complexes on progression of neoplastic diseases can be monitored by any methods known to one skilled in the art, including but not limited to measuring: a) delayed hypersensitivity as an assessment of cellular immunity; b) activity of cytolytic T-lymphocytes in vitro; c) levels of tumor specific antigens, e.g., carcinoembryonic (CEA) antigens; d) changes in the morphology of tumors using techniques such as a computed tomographic (CT) scan; e) changes in levels of putative biomarkers of risk for a particular cancer in individuals at high risk, and f) changes in the morphology of tumors using a sonogram. Other techniques that can also be used include scintigraphy and endoscopy.

The preventive effect of immunotherapy using HPBF-peptide complexes may also be estimated by determining levels of a putative biomarker for risk of a specific cancer. For example, in individuals at enhanced risk for prostate cancer, serum prostate-specific antigen (PSA) is measured by the procedure described by Brawer et al., 1992, J. Urol. 147:841-845, and Catalona et al., 1993, JAMA 270:948-958; or in individuals at risk for colorectal cancer, CEA is measured by methods known in the art; and in individuals at enhanced risk for breast cancer, 16-a-hydroxylation of estradiol is measured by the procedure described by Schneider et al., 1982, Proc. Natl. Acad. Sci. USA 79:3047-3051. The references cited above are incorporated by reference herein in their entirety.

5.4.3 Combination With Adoptive Immunotherapy

Adoptive immunotherapy refers to a therapeutic approach for treating infectious diseases or cancer in which immune cells are administered to a host with the aim that the cells mediate specific immunity, either directly or indirectly, to the infected cells or tumor cells and/or antigenic components, and result in treatment of the infectious disease or regression of the tumor, as the case may be (see U.S. Patent application Ser. No. 08/527,546, filed Sep. 13, 1995, which is incorporated by reference herein in its entirety). The use of stress protein-peptide complexes for sensitizing antigen presenting cells in vitro for use in adoptive immunotherapy is described in PCT publication WO 97/10002, dated Mar. 20, 1997, which is incorporated by reference herein in its entirety. Methods for sensitizing antigen presenting cells (APC) using HPBFs in noncovalently complexes with antigenic (or immunogenic) molecules, for adoptive immunotherapy are described in detail herein.

According to the invention, therapy by administration of HPBF-peptide complexes, using any desired route of administration, is combined with adoptive immunotherapy using APC sensitized with HPBF-antigenic molecule complexes. The HPBF-peptide complex-sensitized APC can be administered concurrently with HPBF-peptide complexes, or before or after administration of HPBF-peptide complexes. Furthermore, the mode of administration can be varied, including but not limited to, e.g., subcutaneously, intravenously, intraperitoneally, intramuscularly, intradermally or mucosally.

5.4.3.1 Sensitization of Macrophages and Antigen Presenting Cells with HSP Peptide-Binding Fragment-Peptide Complexes

The antigen-presenting cells, including but not limited to macrophages, dendritic cells and B-cells, are preferably obtained by production in vitro from stem and progenitor cells from human peripheral blood or bone marrow as described by Inaba et al., 1992, J. Exp. Med. 176:1693-1702. APC can be obtained by any of various methods known in the art. In a preferred aspect human macrophages are used, obtained from human blood cells.

By way of example, but not limitation, macrophages can be obtained as follows: Mononuclear cells are isolated from peripheral blood of a patient (preferably the patient to be treated), by Ficoll-Hypaque gradient centrifugation and are seeded on tissue culture dishes which are pre-coated with the patient's own serum or with other AB+ human serum. The cells are incubated at 37° C. for 1 hr, then non-adherent cells are removed by pipetting. To the adherent cells left in the dish, is added cold (4° C.) 1 mM EDTA in phosphate-buffered saline and the dishes are left at room temperature for 15 minutes. The cells are harvested, washed with RPMI buffer and suspended in RPMI buffer. Increased numbers of macrophages may be obtained by incubating at 37° C. with macrophage-colony stimulating factor (M-CSF); increased numbers of dendritic cells may be obtained by incubating with granulocyte-macrophage-colony stimulating factor (GM-CSF) as described in detail by Inaba, K., et al., 1992, J. Exp. Med. 176:1693-1702.

APC are sensitized with HPBFs noncovalently bound to antigenic molecules by incubating the cells in vitro with the complexes. The APC are sensitized with complexes of HPBF and antigenic molecules preferably by incubating in vitro with the HPBF-complex at 37° C. for 15 minutes to 24 hours. By way of example but not limitation, 4×10⁷ macrophages can be incubated with 10 microgram gp96-peptide complexes per ml or 100 microgram hsp90-peptide complexes per ml at 37° C. for 15 minutes-24 hours in 1 ml plain RPMI medium. The cells are washed three times and resuspended in a physiological medium preferably sterile, at a convenient concentration (e.g., 1×10⁷/ml) for injection in a patient. In a preferred embodiment, the antigen presenting cells are autologous to the patient, that is, the patient into which the sensitized APCs are injected is the patient from which the APC were originally isolated,.

Optionally, the ability of sensitized APC to stimulate, for example, the antigen-specific, class I-restricted cytotoxic T-lymphocytes (CTL) can be monitored by their ability to stimulate CTLs to release tumor necrosis factor, and by their ability to act as targets of such CTLs.

5.4.3.2 Reinfusion of Sensitized APC

The HPBF-antigenic molecule-sensitized APC are reinfused into the patient systemically, preferably intravenously, by conventional clinical procedures. These activated cells are reinfused, preferentially by systemic administration into the autologous patient. Patients generally receive from about 10⁶ to about 10¹² sensitized macrophages, depending on the condition of the patient. In some regimens, patients may optionally receive in addition a suitable dosage of a biological response modifier including but not limited to the cytokines IFN-α, IFN-γ, IL-2, IL-4, IL-6, TNF or other cytokine growth factor.

5.4.4 Determination of Immunogenicity of HSP-Peptide Complexes

In an optional procedure, the purified HPBF-peptide complexes can be assayed for immunogenicity using the mixed lymphocyte target culture assay (MLTC) well known in the art.

By way of example but not limitation, the following procedure can be used.

Briefly, mice are injected subcutaneously with the candidate HPBF-peptide complexes. As a positive control another set of mice are immunized with whole cancer cells of the type from which the HPBFs are derived. As a negative control, mice are injected with either HSP-peptide complexes isolated from normal, non-recombinant cells or whole cells (i.e., antigenically distinct from the type of cell from which the HPBFs are derived). The mice are injected twice, 7-10 days apart. The mice are injected twice, 7-10 days apart. Ten days after the last immunization, the spleens are removed and the lymphocytes released. The released lymphocytes may be restimulated subsequently in vitro by the addition of dead cells that expressed the complex of interest.

For example, 8×10⁶ immune spleen cells may be stimulated with 4×10⁴ mitomycin C treated or γ-irradiated (5-10,000 rads) pathogen-infected cells (or cells transfected with a gene encoding an antigen of the infectious agent, as the case may be), or tumor cells in 3 ml RPMI medium containing 10% fetal calf serum. In certain cases 33% secondary mixed lymphocyte culture supernatant or interleukin 2 (IL-2) may be included in the culture medium as a source of T cell growth factors (See, Glasebrook et al., 1980, J. Exp. Med. 151:876). To test the primary cytotoxic T cell response after immunization, spleen cells may be cultured without stimulation. In some experiments spleen cells of the immunized mice may also be restimulated with antigenically distinct cells, to determine the specificity of the cytotoxic T cell response.

Six days later the cultures are tested for cytotoxicity in a 4 hour ⁵¹Cr-release assay (See, Palladino et al., 1987, Cancer Res. 47:5074-5079 and Blachere, at al., 1993, J. Immunotherapy 14:352-356). In this assay, the mixed lymphocyte culture is added to a target cell suspension to give different effector:target (E:T) ratios (usually 1:1 to 40: 1). The target cells are prelabelled by incubating 1×10⁶ target cells in culture medium containing 200 mCi ⁵¹Cr/ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelleted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5% (Heike et al., 1994, J. Immunotherapy 15:165-174).

An alternative to the chromium-release assay is the ELISPOT assay which measures cytokine release by cytotoxic T cells in vitro after stimulation with specific antigen. Cytokine release is detected by antibodies which are specific for a particular cytokine, such as interleukin-2, tumor necrosis factor αor interferon-γ (for example, see Scheibenbogen et al., 1997, Int. J. Cancer, 71:932-936). The assay is carried out in a microtitre plate which has been pre-coated with an antibody specific for a cytokine of interest which captures the cytokine secreted by T cells. After incubation of T cells for 24-48 hours in the coated wells, the cytotoxic T cells are removed and replaced with a second labelled antibody that recognizes a different epitope on the cytokine. After extensive washing to remove unbound antibody, an enzyme substrate which produces a colored reaction product is added to the plate. The number of cytokine-producing cells is counted under a microscope. This method has the advantages of short assay time, and sensitivity without the need of a large number of cytotoxic T cells.

5.4.5 Monitoring of Effects During Immunotherapy

The effect of immunotherapy with HPBF-antigenic molecule complexes can be monitored by any methods known to one skilled in the art, including but not limited to measuring: a) delayed hypersensitivity as an assessment of cellular immunity; b) activity of cytolytic T-lymphocytes in vitro; c) levels of infective agent-agent or tumor-specific antigens, e.g., carcinoembryonic (CEA) antigens. In the case of the use of HPBF complexes for prevention or treatment of cancer, the effect can additionally be monitored by: d) changes in the morphology of tumors using techniques such as a computed tomographic (CT) scan; and e) changes in levels of putative biomarkers of risk for a particular cancer in individuals at high risk, and f) changes in the morphology of tumors using a sonogram.

5.4.5.1 Delayed Hypersensitivity Skin Test

Delayed hypersensitivity skin tests are of great value in the overall immunocompetence and cellular immunity to an antigen. Inability to react to a battery of common skin antigens is termed anergy (Sato et al., 1995, Clin. Immunol. Pathol. 74:35-43).

Proper technique of skin testing requires that the antigens be stored sterile at 4° C., protected from light and reconstituted shorted before use. A 25- or 27-gauge needle ensures intradermal, rather than subcutaneous, administration of antigen. Twenty-four and 48 hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate test.

5.4.5.2 Activity of Cytolytic T Lymphocytes In Vitro

The activity of cytolytic T-lymphocytes can be assessed in vitro using the following method. Eight×10⁶ peripheral blood-derived T lymphocytes isolated by the Ficoll-Hypaque centrifugation gradient technique, are restimulated with 4×10⁴ mitomycinc-treated tumor cells in 3 ml RPMI medium containing 10% fetal calf serum. In some experiments, 33% secondary mixed lymphocyte culture supernatant or IL-2, is included in the culture medium as a source of T cell growth factors.

In order to measure the primary response of cytolytic T-lymphocytes after immunization, T cells are cultured without the stimulator tumor cells. In other experiments, T cells are restimulated with antigenically distinct cells. After six days, the cultures are tested for cytotoxicity in a 4 hour ⁵¹Cr-release assay. The spontaneous ⁵¹Cr-release of the targets should reach a level less than 20%. For the anti-MHC class I blocking activity, a tenfold concentrated supernatant of W6/32 hybridoma is added to the test at a final concentration of 12.5% (Heike M., et al., J Immunotherapy 15:165-174).

5.4.5.3 Levels of Tumor Specific Antigens

Although it may not be possible to detect unique tumor antigens on all tumors, many tumors display antigens that distinguish them from normal cells. The monoclonal antibody reagents have permitted the isolation and biochemical characterization of the antigens and have been invaluable diagnostically for distinction of transformed from nontransformed cells and for definition of the cell lineage of transformed cells. The best-characterized human tumor-associated antigens are the oncofetal antigens. These antigens are expressed during embryogenesis, but are absent or very difficult to detect in normal adult tissue. The prototype antigen is carcinoembryonic antigen (CEA), a glycoprotein found on fetal gut and human colon cancer cells, but not on normal adult colon cells. Since CEA is shed from colon carcinoma cells and found in the serum, it was originally thought that the presence of this antigen in the serum could be used to screen patients for colon cancer. However, patients with other tumors, such as pancreatic and breast cancer, also have elevated serum levels of CEA. Therefore, monitoring the fall and rise of CEA levels in cancer patients undergoing therapy has proven useful for predicting tumor progression and responses to treatment.

Several other oncofetal antigens have been useful for diagnosing and monitoring human tumors, e.g., alpha-fetoprotein, an alpha-globulin normally secreted by fetal liver and yolk sac cells, is found in the serum of patients with liver and germinal cell tumors and can be used as a marker of disease status.

5.4.5.4 Computed Tomographic (CT) Scan

CT remains the choice of techniques for the accurate staging of cancers. CT has proved more sensitive and specific than any other imaging techniques for the detection of metastases.

5.4.5.5 Measurement of Putative Biomarkers

The levels of a putative biomarker for risk of a specific cancer are measured to monitor the effect of hsp noncovalently bound to peptide complexes. For example, in individuals at enhanced risk for prostate cancer, serum prostate-specific antigen (PSA) is measured by the procedure described by Brawer, M. K., et. al., 1992, J. Urol. 147:841-845, and Catalona, W. J., et al., 1993, JAMA 270:948-958; and in individuals at enhanced risk for breast cancer, 16-a-hydroxylation of estradiol is measured by the procedure described by Schneider, J. et al, 1982, Proc. Natl. Acad. Sci. ISA 79:3047-3051.

5.4.5.6 Sonogram

A sonogram remains an alternative choice of technique for the accurate staging of cancers.

5.5 Dosage Regimens

It was established in experimental tumor models (Blachere et al, 1993, J Immunotherapy 14:352-356) that the lowest dose of HSP noncovalently bound to peptide complexes which produced tumor regression in mice was between 10 and 25 microgram/mouse weighing 20-25 g which is equal to 25 mg/25 g=1 mg/kg. These amounts should be decreased proportionally with the decreased molecular weight of the HSP binding domain fragments.

Prior art methods extrapolate to human dosages based on body weight and surface area. For example, prior art methods of extrapolating human dosage based on body weight can be carried out as follows: since the conversion factor for converting the mouse dosage to human dosage is Dose Human per kg=Dose Mouse per kg×12 (See Freireich, E. J., et al., 1966, Cancer Chemotherap. Rep. 50:219-244), the effective dose of HSP-peptide complexes in humans weighing 70 kg should be 1 mg/kg÷12×70, i.e., about 6 mg (5.8 mg).

Drug doses are also given in milligrams per square meter of body surface area because this method rather than body weight achieves a good correlation to certain metabolic and exeretionary functions (Shirkey, 1965, JAMA 193:443). Moreover, body surface area can be used as a common denominator for drug dosage in adults and children as well as in different animal species as indicated below in Table 1 (Freireich et al, 1966, Cancer Chemotherap. Rep. 50:219-244).

TABLE 1
REPRESENTATIVE SURFACE AREA TO WEIGHT
RATIOS (km) FOR VARIOUS SPECIES¹

| | Body Weight | Surface Area | |
|---|---|---|---|
| Species | (kg) | (Sqm) | km Factor |
| Mouse | 0.02 | 0.0066 | 3.0 |
| Rat | 0.15 | 0.025 | 5.9 |
| Monkey | 3.0 | 0.24 | 12 |
| Dog | 8.0 | 0.40 | 20 |
| Human, Child | 20 | 0.80 | 25 |
| Adult | 60 | 1.6 | 37 |
| Example: To express a mg/kg dose in any given species as the equivalent mg/sq m dose, multiply the dose by the | | | |
| # appropriate km factor. In adult human, 100 mg/kg is equivalent to 100 mg/kg × 37 kg/sq m = 3700 mg/sq m. | | | |

In contrast to both of the above-described prior art methods of determining dosage levels, the present invention provides dosages of the purified complexes of fragments HSPs and antigenic molecules that are much smaller than the dosages estimated by the prior art. For example, according to the invention, an amount of Hsp70fragment-antigenic molecule complexes and/or gp96 fragment-antigenic molecule complexes is administered that is in the range of about 2 microgram to about 150 micrograms for a human patient, the preferred human dosage being the same as used in a 25 g mouse. The dosage for Hsp-90 peptide complexes in a human patient provided by the present invention is in the range of about 10 to 1,000 micrograms, the preferred dosage being 20 micrograms.

The doses recited above are preferably given once weekly for a period of about 4-6 weeks, and the mode or site of administration is preferably varied with each administration. In a preferred example, subcutaneous administrations are given, with each site of administration varied sequentially. Thus, by way of example and not limitation, the first injection may be given subcutaneously on the left arm, the second on the right arm, the third on the left belly, the fourth on the right belly, the fifth on the left thigh, the sixth on the right thigh, etc. The same site may be repeated after a gap of one or more injections. Also, split injections may be given. Thus, for example, half the dose may be given in one site and the other half on an other site on the same day.

Alternatively, the mode of administration is sequentially varied, e.g., weekly injections are given in sequence subcutaneously, intramuscularly, intravenously or intraperitoneally.

After 4-6 weeks, further injections are preferably given at two-week intervals over a period of time of one month. Later injections may be given monthly. The pace of later injections may be modified, depending upon the patient's clinical progress and responsiveness to the immunotherapy.

The invention is illustrated by non-limiting examples in Sections 6 and 7.

5.6 Formulation

Noncovalent complexes of HPBFs and antigenic proteins or peptides purified by the methods of the invention may be formulated into pharmaceutical preparations for administration to mammals for treatment or prevention of infectious diseases or cancer. Drug solubility and the site of absorption are factors which should be considered when choosing the route of administration of a therapeutic agent.

HPBF-antigenic molecule complexes of the invention may optionally be administered with one or more adjuvants in order to enhance the immunological response. For example, depending on the host species, adjuvants which may be used include, but are not limited to: mineral salts or mineral gels such as aluminum hydroxide, aluminum phosphate, and calcium phosphate; surface active substances such as lysolecithin, pluronic polyols, polyanions, peptides, oil emulsions, keyhole limpet hemocyanins, and dinitrophenol; immunostimulatory molecules, such as cytokines, saponins, muramyl dipeptides and tripeptide derivatives, CpG dinucleotides, CpG oligonucleotides, monophosphoryl Lipid A, and polyphosphazenes; particulate and microparticulate adjuvant, such as emulsions, liposomes, virosomes, cochleates; or an immune stimulating complex mucosal adjuvants, Freund's (complete and incomplete, and potentially useful human adjuvants such as BCG (bacille Calmette-Guerin) and corynebacterium parvum.).

HPBF-antigenic molecule complexes of the invention may be administered using any desired route of administration, including but not limited to, e.g., subcutaneously, intravenously or intramuscularly, although intradermally or mucosally is preferred. Advantages of intradermal or mucosal administration include use of lower doses and rapid absorption, respectively. Mucosal routes of administration include, but are not limited to, oral, rectal and nasal administration. Preparations for mucosal administrations are suitable in various formulations as described below. The route of administration can be varied during a course of treatment. Preferred dosages, routes of administration and therapeutic regimens for complexes of peptides and naturally occurring HSPs are described in PCT International patent applications published as WO 96/10411 and WO 97/10001, which are incorporated by reference herein in their entireties.

Compositions comprising noncovalent complexes formulated in a compatible pharmaceutical carrier may be prepared, packaged, and labeled for treatment of the indicated infectious disease or tumor. In preferred aspects, an amount of HPBF complex is administered to a human that is in the range of about 2 to 150 μg, preferably 20 to 20 μg, most preferably about 5 μg, given once weekly for about 4-6 weeks, intradermally with the site of administration varied sequentially.

If the complex is water-soluble, then it may be formulated in an appropriate buffer, for example, phosphate buffered saline or other physiologically compatible solutions. Alternatively, if the resulting complex has poor solubility in aqueous solvents, then it may be formulated with a non-ionic surfactant such as Tween, or polyethylene glycol. Thus, the noncovalent complexes and their physiologically acceptable solvates may be formulated for administration by inhalation or insufflation (either through the mouth or the nose) or oral, buccal, parenteral, rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the complexes. Such compositions may take the form of tablets or lozenges formulated in conventional manner.

For administration by inhalation, the complexes may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the complexes and a suitable powder base such as lactose or starch.

The complexes may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder 1 form for constitution with a suitable vehicle, e.g., sterile pyrogen-free water, before use.

The complexes may also be formulated in rectal compositions such as suppositories or retention enemas, e.g., containing conventional suppository bases such as cocoa butter or other glycerides.

In addition to the formulations described previously, the complexes may also be formulated as a depot preparation. Such long acting formulations may be administered by implantation (for example, subcutaneously or intramuscularly) or by intramuscular injection. Thus, for example, the complexes may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The complexes may, if desired, be presented in a pack or dispenser device which may contain one or more unit dosage forms containing the noncovalent complexes.

The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the noncovalent HPBF-peptide complexes in pharmaceutically acceptable form. The HPBF-peptide complexes in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the complex, and/or a packaged alcohol pad. Instructions are optionally included for administration of HPBF-peptide complexes by a clinician or by the patient.

### 6. EXAMPLE

Administration of Hsp Peptide-Binding Fragment-Peptide Complexes in the Treatment of Infectious Disease

Recombinant cells are engineered to express a peptide-binding domain of Hsp70 and either: peptide A, KRQITDLEMNRLGK (SEQ ID NO:9), derived from G protein of vesicular stomatitis virus (VSV); peptide B, LSSLFRPKRRPIYKS (SEQ ID NO:10) (derived from VSV G protein); peptide C, SLSDLRGYVYQGLKSGNVS (SEQ ID NO: 11) (derived from VSV nucleoprotein; see Blachere et al, J. Exp. Med. 1997, 186: 1315-1322; Flynn et al., 1989, Science 245:385-90). Mice, or other subject, with VSV are injected with HPBF-peptide complexes derived from such cells, either separately or in combination. The Hsp70 peptide-binding fragment includes amino acid residues a region from the extreme carboxy-terminus of Hsp70, encompassing amino acid residues 391 to 615. The therapeutic regiment of Hsp70 fragment-peptide complexes, includes weekly injections of the Hsp70 fragment-peptide complexes, dissolved in saline or other physiologically compatible solution.

The dosage used for Hsp70 peptide-binding fragment complexes is in the range of 2-150 micrograms, with the preferred dosage being 2-20 micrograms. The dosage used for Hsp90 peptide-binding fragment complexes is in the range of 10 to 1,000 micrograms, with the preferred dosage being about 20 micrograms.

The route and site of injection is varied each time, for example, the first injection is given subcutaneously on the left arm, the second injection on the right arm, the third injection on the left abdominal region, the fourth injection on the right abdominal region, the fifth injection on the left thigh, the sixth injection on the right thigh, etc. The same site is repeated after a gap of one or more injections. In addition, injections are split and each half of the dose is administered at a different site on the same day.

Overall, the first four to six injections are given at weekly intervals. Subsequently, two injections are given at two-week intervals; followed by a regimen of injections at monthly intervals. The effect of Hsp70 fragment-peptide complex therapy is monitored by measuring: a) delayed hypersensitivity as an assessment of cellular immunity; b) activity of cytolytic T-lymphocytes in vitro; c) levels of viral specific antigens; d) changes in the progression of the VSV disease, using techniques such as a computed tomographic (CT) scan.

Depending on the results obtained, as described above Section 5.5, the therapeutic regimen is developed to maintain and/or boost the immunological responses of the subject, with the ultimate goal of achieving complete eradication of the virus and its symptoms.

### 7. EXAMPLE

Adoptive Transfer of Sensitized Macrophage, in Combination with Administration of Peptide-Binding HSP Peptide-Binding Fragment-Peptide Complexes

Autologous human macrophages are sensitized with the peptide-binding domain of the autologous human Hsp70, noncovalently bound to an antigenic/immunogenic molecule. The peptide-binding domain includes amino acid residues a region from the extreme carboxy-terminus of Hsp70, encompassing amino acid residues 391 to 615. The sensitized macrophages are administered to the human patient at approximately the same time as, or before, or after the administration of the Hsp70 fragment-antigenic molecule complex.

7.1. Materials and Methods

Macrophages are obtained as follows: mononuclear cells are isolated from peripheral blood of the human patient to be treated, by Ficoll-Hypaque gradient centrifugation and are seeded on tissue culture dishes which are pre-coated with the patient's own serum or with other AB+human serum. The cells are incubated at 37° C. for 1 hour, then non-adherent cells are removed by pipetting. To the adherent cells left in the dish, is added cold (40° C.) 1 mM EDTA in phosphate-buffered saline and the dishes are left at room temperature for 15 minutes. The cells are harvested, washed with RPMI buffer and suspended in RPMI buffer. Increased numbers of macrophages may be obtained by incubating at 37° C. with macrophage-colony stimulating factor (M-CSF); increased numbers of dendritic cells may be obtained by incubating with granulocyte-macrophage-colony stimulating factor (GM-CSF) as described in detail by Inaba et al., 1992, J. Exp. Med. 176:1693-1702.

The macrophages (4×10⁷) are then incubated at 37° C. for 3 hr in 1 ml RPMI containing 50 μg Hsp70 fragment-peptide complexes derived from the autologous tumor or from autologous liver, using methods as described in Section 5.3.1.2. The macrophages are then washed 3 times and resuspended at a concentrate of 1×10⁷/ml in RPMI medium. Two hundred microliters of this suspension is administered as described in the experimental protocol below.

7.2. Treatment of Hepatocellular Carcinoma

Five groups of human patients with hepatocellular carcinoma are injected with autologous macrophages sensitized with Hsp70 fragment-peptide complexes derived from their own tumors post surgery. Treatment with Hsp70 fragment-peptide complexes is started any time after surgery. However, if the patient has received chemotherapy, sensitized macrophages alone or in combination with HPBF-peptide complexes are administered after an interval of four weeks or more so as to allow the immune system to recover. The immunocompetence of the patient is tested by procedures described in Sections 5.4.5, above.

The preferred therapeutic regimen includes weekly injections of the sensitized macrophages in combination with an Hsp-peptide complex dissolved in saline or other physiologically compatible solution. Sensitized macrophages may be administered at approximately the same time with an hsp-peptide complex or one may be administered prior to administration of the other.

The dosage used for Hsp70 fragments or gp96 fragments is in the range of 0.1 to 9 micrograms, with the preferred dosage being 0.5-2.0 micrograms. The dosage used for Hsp90 fragments is in the range of 1 to 100 micrograms, with the preferred dosage being about 2 micrograms.

The site of injection is varied each time, for example, the first injection is given intradermally on the left arm, the second injection intradermally on the right arm, the third injection intradermally on the left abdominal region, the fourth injection intradermally on the right abdominal region, the fifth injection intradermally on the left thigh, the sixth injection intradermally on the right thigh, etc. The same site can be alternatively repeated after a gap of one or more injections. In addition, injections are split and each half of the dose is administered at a different site on the same day.

Overall, the first four to six injections are given at weekly intervals. Subsequently, two injections are given at two-week intervals; followed by a regimen of injections at monthly intervals. The effect of therapy is monitored by measuring: a) delayed hypersensitivity as an assessment of cellular immunity; b) activity of cytolytic T-lymphocytes in vitro; c) levels of tumor specific antigens, e.g., carcinoembryonic (CEA) antigens; d) changes in the morphology of tumors using techniques such as a computed tomographic (CT) scan; and/or e) changes in putative biomarkers of risk for a particular cancer in individuals at high risk.

Depending on the results obtained, as described above in Section 5.4.1, the therapeutic regimen may be modified to maintain and/or boost the immunological responses of the patient, with the ultimate goal of achieving tumor regression and complete eradication of cancer cells.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes.

What is claimed is:

1. A pharmaceutical composition comprising an amount of a molecular complex effective for treatment or prevention of an infectious disease or cancer, and a pharmaceutically acceptable carrier, said molecular complex comprising a heat shock protein peptide-binding fragment noncovalently associated with an antigenic molecule, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, and wherein said antigenic molecule displays the antigenicity of an antigen of an infectious agent or of a cancer cell.

2. The pharmaceutical composition of claim 1 wherein the heat shock protein is Hsp70, Hsp90, gp96, calreticulin, or PDI.

3. The pharmaceutical composition of claim 2 wherein the heat shock protein is Hsp70.

4. The pharmaceutical composition of claim 2 wherein the heat shock protein is Hsp90.

5. The pharmaceutical composition of claim 2 wherein the heat shock protein is PDI.

6. The pharmaceutical composition of claim 2 wherein the heat shock protein is gp96.

7. The pharmaceutical composition of claim 1 wherein the molecular complex is purified.

8. The pharmaceutical composition of claim 1 wherein said heat shock protein fragment lacks one or more other domains of the heat shock protein.

9. A recombinant cell infected with a pathogen and transformed with a nucleic acid comprising a nucleotide sequence that (i) is operably linked to a promoter, and (ii) encodes a heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, which heat shock protein peptide-binding fragment noncovalently associates with an antigenic molecule when said antigenic molecule is present, to form a complex that in sufficient amount is capable of eliciting an immune response to the antigenic molecule.

10. The recombinant cell of claim 9 wherein said cell is a human cell.

11. A recombinant cancer cell transformed with a nucleic acid comprising a nucleotide sequence that (i) is operably linked to a promoter, and (ii) encodes a heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, which heat shock protein peptide-binding fragment noncovalently associates with an antigenic molecule when said antigenic molecule is present, to form a complex that in sufficient amount is capable of eliciting an immune response to the antigenic molecule.

12. The recombinant cancer cell of claim 9 or 11 wherein the cell is a human cell.

13. A recombinant cell transformed with (i) a first nucleic acid comprising a first nucleotide sequence that is operably linked to a first promoter and that encodes a heat shock protein peptide-binding fragment a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, and (ii) a second nucleic acid comprising a second nucleotide sequence that is operably linked to a second promoter and encodes an antigenic molecule, such that the heat shock protein peptide-binding fragment and the antigenic molecule are expressed within the cell and noncovalently associate with each other to form a complex that in sufficient amount is capable of eliciting an immune response to the antigenic molecule.

14. A pharmaceutical composition comprising the recombinant cell of claim 13 wherein said heat shock protein fragment lacks one or more other domains of the heat shock protein.

15. A pharmaceutical composition comprising the recombinant cell of any one of claims 9, 11, or 13 and a pharmaceutically acceptable carrier.

16. A method for preparing a complex of a heat shock protein peptide-binding fragment noncovalently associated with a peptide, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, comprising:
a) culturing cells, transformed with a nucleic acid comprising a nucleotide sequence encoding the heat shock protein peptide-binding fragment and operably linked to a promoter, under conditions such that the heat shock protein peptide-binding fragment is expressed by the cells and associates with peptides of the cells; and
b) recovering a population of complexes of the heat shock protein peptide-binding fragment noncovalently associated with peptides from the cells.

17. A method for preparing a heat shock protein peptide-binding fragment noncovalently associated with peptides derived from one or more antigens of an infectious agent, said heat shock protein peptide-binding fragment comprising a peptide-binding domain that is contiguous on the N-termninal side with x number of amino acids that naturally flank the peptide-binding domain on the N-terminal side, and that is contiguous on the C-termninal side with y number of amino acids that naturally flank the peptide-binding domain on the C-termninal side, wherein x plus y is not more than 400, comprising:
a) culturing infected cells, transformed with a nucleic acid comprising a nucleotide sequence encoding the heat shock protein peptide-binding fragment and operably linked to a promoter, under conditions such that the heat shock protein peptide-binding fragment is expressed by the cells and associates with peptides of the cells; and b) recovering from the cells a population of complexes of the heat shock protein peptide-binding fragment noncovalently associated with peptides derived from the infectious agent.

18. A method for preparing a complex of a heat shock protein peptide-binding fragment noncovalently associated with a peptide, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said method comprising digesting a preparation of heat shock proteins noncovalently associated with peptides with a protease under conditions and for a length of time sufficient for the formation of peptide-binding fragments of the heat shock protein noncovalently associated with peptides.

19. A method for preparing a complex of a heat shock protein peptide-binding fragment noncovalently associated with a peptide, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said method comprising:
a) digesting a preparation of heat shock proteins with a protease under conditions and for a length of time sufficient for the formation of peptide-binding fragments of the heat shock protein; and
b) contacting the peptide-binding fragments with peptides under conditions and for a length of time sufficient for the formation of complexes of heat shock protein peptide-binding fragments noncovalently associated with peptides.

20. The method of any one of claims 16-19, further comprising purifying the complexes.

21. The method of any one of claims 16-19, further comprising purifying the complexes by affinity chromatography.

22. A method for preparing in vitro complexes of heat shock protein peptide-binding fragments noncovalently associated with one or more antigenic molecules, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said method comprising incubating a heat shock protein peptide-binding fragment and one or more antigenic molecules under conditions and for a length of time sufficient for the formation of the complexes.

23. The method of claim 22 wherein the one or more antigenic molecules is a population of peptides from an infected cell or a cancer cell.

24. The method of claim 22 wherein the one or more antigenic molecules displays the antigenicity of an antigen of an infectious agent or a cancer cell.

25. A method of eliciting an immune response against an antigen in an individual comprising administering to the individual an immunogenic complex of a heat shock protein peptide-binding fragment noncovalently associated with a first antigenic molecule displaying antigenicity of the antigen, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400.

26. The method of claim 25, wherein said heat shock protein fragment lacks one or more other domains of the heat shock protein.

27. The method of claim 25, further comprising, before, concurrently, or after administration of the immunogenic complex, administering to the individual a composition comprising antigen presenting cells sensitized in vitro with a sensitizing amount of a second immunogenic complex consisting essentially of a heat shock protein, or peptide-binding fragment thereof, noncovalently bound to a second antigenic molecule, in which said second antigenic molecule shares at least one antigenic determinant with the first antigenic molecule.

28. A method of treating or preventing an infectious disease in an individual having an infectious disease, or in whom prevention of an infectious disease is desired, comprising administering to the individual an immunogenic complex of a heat shock protein peptide-binding fragment noncovalently associated with a first antigenic molecule, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, wherein the first antigenic molecule displays the antigenicity of an antigen of an infectious agent of the infectious disease.

29. The method of claim 28, wherein said heat shock protein fragment lacks one or more other domains of the heat shock protein.

30. The method of claim 28, further comprising, before, concurrently or after administration of the immunogenic complex, administering to the individual a composition comprising antigen presenting cells sensitized in vitro with a sensitizing amount of a second complex of a heat shock protein or peptide-binding fragment thereof noncovalently bound to a second antigenic molecule, said second antigenic molecule sharing at least one antigenic determinant with the first antigenic molecule.

31. A method of treating or preventing an infectious disease in a subject having an infectious disease or in whom prevention of an infectious disease is desired comprising:
a) culturing an infected cell transformed with a nucleic acid comprising a nucleotide sequence encoding a heat shock protein peptide-binding fragment, said infected cell displaying the antigenicity of an antigen of an infectious agent of the infectious disease, said heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said nucleotide sequence being operably linked to a promoter, under conditions such that the peptide-binding fragment is expressed by the infected cells and associates with peptides of the cell;
b) recovering complexes of the heat shock protein peptide-binding fragments noncovalently associated with peptides from the infected cell; and
c) administering to the subject an amount of the recovered complexes effective to treat or prevent the infectious disease.

32. The method of claim 31, wherein said heat shock protein fragment lacks one or more other domains of the heat shock protein.

33. The method of claim 31, further comprising, prior to step (a), the step of obtaining infected cells from the subject and transforming the infected cells with the nucleic acid.

34. The method of claim 31, further comprising, prior to step (a), the step of obtaining the infected cell from one or more individuals and transforming the infected cells with the nucleic acid, said one or more individuals being different from the subject and having the same type of infectious disease as the subject.

35. A method of treating or preventing an infectious disease in a subject having an infectious disease or in whom prevention of an infectious disease is desired comprising:
a) culturing a recombinant cell transformed with (i) a first nucleic acid encoding a heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, and (ii) a second nucleic acid encoding an antigenic molecule displaying the antigenicity of an antigen of an infectious agent of the infectious disease;
b) recovering complexes of the heat shock protein peptide-binding fragments noncovalently associated with the antigenic molecule; and
c) administering to the subject an amount of the recovered complexes effective to treat or prevent the infectious disease.

36. The method of claim 28, 31, or 35, in which the infectious disease is caused by an infectious agent selected from the group consisting of a virus, a bacterium, a fungus, and a parasite.

37. A method of treating or preventing cancer in an individual having a type of cancer or in whom prevention of a type of cancer is desired comprising administering to the individual an immunogenic complex of a heat shock protein peptide-binding fragment noncovalently associated with a first antigenic molecule, said heat shock protein comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, wherein either (a) the first antigenic molecule displays antigenicity of said type of cancer or a metastasis thereof; or (b) the complex is obtained by recovering complexes from said type of cancer cells or a metastasis thereof that recombinantly express the heat shock protein peptide-binding fragment.

38. The method of claim 37, further comprising, before, concurrently or after administration of the immunogenic complex, administering to the individual a composition comprising antigen presenting cells sensitized in vitro with a sensitizing amount of a second complex of a heat shock protein or peptide-binding fragment thereof noncovalently bound to a second antigenic molecule, said second antigenic molecule sharing at least one antigenic determinant with the first antigenic molecule.

39. A method of treating or preventing cancer in a subject having a type of cancer or in whom prevention of a type of cancer is desired comprising:
a) culturing a cancer cell transformed with a nucleic acid comprising a nucleotide sequence encoding a heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, said nucleotide sequence being operably linked to a promoter, under conditions such that the peptide-binding fragment is expressed by the cancer cell and associates with peptides of the cell;
b) recovering complexes of the heat shock protein peptide-binding fragments noncovalently associated with peptides from the cancer cell; and
c) administering to the subject an amount of the recovered complexes effective to treat or prevent cancer.

40. The method of claim 39, further comprising, prior to step (a), the step of obtaining cancer cells from the subject and transforming the cancer cells with the nucleic acid.

41. The method of claim 39, further comprising, prior to step (a), the step of obtaining cancer cells from one or more individuals and transforming the cancer cells with the nucleic acid, said one or more individuals being different from the subject and having the same type of cancer as the subject.

42. A method of treating or preventing cancer in a subject having a type of cancer or in whom prevention of a type of cancer is desired comprising:
a) culturing a recombinant cell transformed with (i) a first nucleic acid encoding a heat shock protein peptide-binding fragment comprising a peptide-binding domain that is contiguous on the N-terminal side with x number of amino acids that naturally flank the peptide-binding domain on the N-terminal side, and that is contiguous on the C-terminal side with y number of amino acids that naturally flank the peptide-binding domain on the C-terminal side, wherein x plus y is not more than 400, and (ii) a second nucleic acid encoding an antigenic molecule displaying the antigenicity of an antigen of a cancer cell;
b) recovering complexes of the heat shock protein peptide-binding fragments noncovalently associated with the antigenic molecule; and
c) administering to the subject an amount of the recovered complexes effective to treat or prevent cancer.

43. The method of claim 25, 28, 31, 35, 37, 39, or 42, wherein the peptide-binding domain consists of the peptide-binding domain of Hsp70, Hsp90, gp96, calreticulin, PDI, or a mixture of two or more of the foregoing.

44. The method of claim 43, wherein the heat shock protein peptide-binding fragment is a human gp96 peptide-binding fragment comprising the amino acid sequence from about position 5 to about position 232 of the amino acid sequence shown in FIG. 2C (SEQ ID NO:7).

45. The method of claim 43, wherein the heat shock protein peptide-binding fragment is a gp96 peptide-binding fragment comprising the amino acid sequence from about position 615 to about position 658 of the amino acid sequence shown in FIG. 2C (SEQ ID NO:7).

46. The method of claim 43, wherein the heat shock protein peptide-binding fragment is a gp96 peptide-binding fragment comprising the amino acid sequence from about position 624 to about position 630 of the amino acid sequence shown in FIG. 2C (SEQ ID NO:7).
